(19) United States
(12) Patent Application Publication
(10) Pub. No.: US 2026/0207106 A1
(43) Pub. Date: Jul. 23, 2026

(54) DEVICES FOR MEASURING BRAIN SIGNALS

(71) Applicant: Electronics and Telecommunications Research Institute, Daejeon (KR)

(72) Inventors: Chang Han JE, Daejeon (KR); Sungryul YUN, Daejeon (KR); Hyung-Kun LEE, Daejeon (KR)

(73) Assignee: Electronics and Telecommunications Research Institute, Daejeon (KR)

(21) Appl. No.: 19/225,493

(22) Filed: Jun. 2, 2025

(30) Foreign Application Priority Data
Jan. 17, 2025 (KR) 10-2025-0007564

(51) Int. Cl. A61B 5/293; A61B 5/268; A61B 5/369

(52) U.S. Cl. CPC A61B 5/293; A61B 5/268; A61B 5/369; A61B 2562/04; A61B 2562/164

(57) ABSTRACT

Provided is a device for measuring brain signals, comprising an electrode substrate comprising a first surface and a second surface opposing each other, a plurality of flexible electrodes extending in a direction perpendicular to the first surface of the electrode substrate on the electrode substrate, and a polymer layer covering at least a portion of a side surface of each of the plurality of flexible electrodes, wherein each of the plurality of flexible electrodes comprisies a plurality of sensing portions, the polymer layer has greater mechanical properties at a temperature of 25 °C than at a temperature of 36 °C, and the mechanical properties are storage modulus.

### CROSS-REFERENCE TO RELATED APPLICATIONS

This U.S. non-provisional patent application claims priority under 35 U.S.C. § 119 of Korean Patent Application No. 10-2025-0007564, filed on January 17, 2025, the entire contents of which are hereby incorporated by reference.

### BACKGROUND

The present disclosure relates to a device for measuring brain signals, and more particularly, to a device for measuring brain signals coated with a polymer whose stiffness changes depending on temperature.

Recently, with the growing interest in the brain, active research is underway to stimulate brain nerves or analyze brain signals for the treatment of brain-related diseases or for the rehabilitation of related disorders. Initially, attempts were made to indirectly analyze brain nerves and signals using electricity, ultrasound, heat, and light from outside the body to analyze the nerves and signals of the brain. However, the information obtainable through these indirect methods was limited.

Recently, invasive brain implant electrode technologies designed to directly access the brain to acquire brain signals and stimulate brain nerves are on the rise. However, these invasive brain implant electrodes cause inflammation or damage due to continuous stimulation of brain tissue.

### SUMMARY

The present disclosure provides a device for measuring brain signals, which measures brain signals while minimizing damage to brain tissue.

An embodiment of the inventive concept provides a device for measuring brain signals, including an electrode substrate including a first surface and a second surface opposing each other, a plurality of flexible electrodes extending in a direction perpendicular to the first surface of the electrode substrate on the electrode substrate, and a polymer layer covering at least a portion of a side surface of each of the plurality of flexible electrodes, wherein each of the plurality of flexible electrodes includes a plurality of sensing portions, the polymer layer has greater mechanical properties at a temperature of about 25 °C than at a temperature of about 36 °C, and the mechanical properties are storage modulus.

In an embodiment of the inventive concept, a device for measuring brain signals includes an electrode substrate, a plurality of flexible electrodes extending in a direction perpendicular to a first surface of the electrode substrate on the electrode substrate, and a polymer layer covering at least a portion of a side surface of each of the plurality of flexible electrodes, wherein each of the plurality of flexible electrodes includes a plurality of sensing portions, the polymer layer has a glass transition temperature of about 34 °C to about 40 °C, and the polymer layer changes from crystalline to amorphous at or above the glass transition temperature.

### BRIEF DESCRIPTION OF THE FIGURES

The accompanying drawings are included to provide a further understanding of the inventive concept, and are incorporated in and constitute a part of this specification. The drawings illustrate embodiments of the inventive concept and, together with the description, serve to explain principles of the inventive concept. In the drawings:

FIG. 1 is a view showing a device for measuring brain signals according to embodiments of the inventive concept;

FIGS. 2 to 5 are views showing a method for manufacturing a device for measuring brain signals according to embodiments of the inventive concept;

FIGS. 6 and 7 are views showing an insertion process of a device for measuring brain signals according to embodiments of the inventive concept;

FIGS. 8 and 9 are views showing a state in which a device for measuring brain signals according to embodiments of the inventive concept is inserted into the brain;

FIG. 10 is a view showing a device for measuring brain signals according to some embodiments of the inventive concept;

FIGS. 11 to 14 are views showing a method for manufacturing a device for measuring brain signals according to embodiments of the inventive concept;

FIG. 15 is a view showing a device for measuring brain signals according to some embodiments of the inventive concept;

FIGS. 16 to 19 are views showing a method for manufacturing a device for measuring brain signals according to embodiments of the inventive concept;

FIG. 20 is a view showing a device for measuring brain signals according to some embodiments of the inventive concept;

FIGS. 21 to 24 are views showing a method for manufacturing a device for measuring brain signals according to embodiments of the inventive concept;

FIG. 25 is a cross-sectional view showing a cross-section of a device for measuring brain signals according to some embodiments of the inventive concept; and

FIG. 26 is a graph showing the results of measuring the stiffness of a polymer according to embodiments of the inventive concept.

### DETAILED DESCRIPTION

Hereinafter, the present disclosure will be described in detail by describing embodiments of the inventive concept with reference to attached drawings.

FIG. 1 is a view showing a device for measuring brain signals according to embodiments of the inventive concept.

Referring to FIG. 1, a device for measuring brain signals 10 may include an electrode substrate 100, a plurality of flexible electrodes 120 on the electrode substrate 100, and a polymer layer 130 covering the plurality of flexible electrodes 120.

The electrode substrate 100 may include a first surface 100a and a second surface 100b opposing each other. A plurality of circuits may be disposed within the electrode substrate 100. The plurality of circuits may be electrically connected to the plurality of flexible electrodes 120. The intensity of brain signals and the type of brain signals may be calculated through the plurality of circuits.

The electrode substrate 100 may include a plurality of electrode connection portions 110 passing through the second surface 100b of the electrode substrate 100. The plurality of electrode connection portions 110 may be spaced apart in a first direction D1. The first direction D1 may indicate a direction parallel to the first surface 100a of the electrode substrate 100. Each of the plurality of electrode connection portions 110 may be mechanically coupled to a corresponding flexible electrode among the plurality of flexible electrodes 120. Each of the plurality of electrode connection portions 110 may apply tension to the coupled flexible electrode, and thus the flexible electrode may be fixed.

The plurality of flexible electrodes 120 may be spaced apart in the first direction D1. Each of the plurality of flexible electrodes 120 may be disposed on the first surface 100a of the electrode substrate 100. Each of the plurality of flexible electrodes 120 may extend in a third direction D3 perpendicular to the first surface 100a. A length 120H of each of the plurality of flexible electrodes 120 in the third direction D3 may be different. Accordingly, a sensing portion 140, which will be described later, may be disposed at various locations. Therefore, signals occurring in various regions of the brain may be measured simultaneously. In FIG. 1, three flexible electrodes 120 are shown, but the inventive concept is not limited thereto, and more than three flexible electrodes 120 may be provided.

The plurality of flexible electrodes 120 may include, for example, a conductive material. Each of the plurality of flexible electrodes 120 may have mechanical properties of less than about 3 MPa, preferably less than about 1 MPa. Herein, the mechanical properties may refer to storage modulus. More specifically, each of the plurality of flexible electrodes 120 may have mechanical properties of less than about 3 MPa, preferably less than about 1 MPa, at a temperature of about 25 °C. Each of the plurality of flexible electrodes 120 may have mechanical properties of less than about 3 MPa, preferably less than about 1 MPa, at a temperature of about 36.5 °C or higher.

When each of the plurality of flexible electrodes 120 has mechanical properties of greater than about 3 MPa at a temperature of about 36.5 °C or higher, brain damage may occur. Each of the plurality of flexible electrodes 120 has greater mechanical properties than the brain, and thus brain damage may occur. Therefore, when each of the plurality of flexible electrodes 120 has mechanical properties of less than about 3 Mpa, brain damage may be prevented.

Each of the plurality of flexible electrodes 120 may have a thickness 120D of, for example, about 5 μm to about 15 μm in the first direction D1. When each of the plurality of flexible electrodes 120 has a thickness 120D of less than about 5 μm in the first direction D1, a surface area in contact with the brain may be small, making it difficult to accurately measure brain signals. In addition, when the thickness 120D is greater than about 15 μm, each of the plurality of flexible electrodes 120 may have greater mechanical properties than the brain, causing brain damage. The plurality of flexible electrodes 120 may include, for example, at least any one selected from the group consisting of graphene, carbon nanotubes, silver nanolines, and copper nanolines.

Each of the plurality of flexible electrodes 120 may include a sensing portion 140. The sensing portion 140 may come into contact with the brain and measure signals occurring in the brain. The sensing portion 140 may be disposed, for example, at an end of each of the plurality of flexible electrodes 120, but is not limited thereto. The length 120H of each of the plurality of flexible electrodes 120 in the third direction D3 is different, and thus the sensing portion 140 may be provided at various locations. Accordingly, signals occurring in various regions of the brain may be measured simultaneously.

The polymer layer 130 may cover the first surface 100a of the electrode substrate 100. The polymer layer 130 may cover at least a portion of a side surface 120S of each of the plurality of flexible electrodes 120. The polymer layer 130 covers the plurality of flexible electrodes 120, and may thus minimize direct contact between the brain and a metal. The polymer layer 130 may expose the sensing portion 140 of each of the plurality of flexible electrodes 120. Accordingly, the sensing portion 140 may come into contact with the brain. The polymer layer 130 covers a side surface of each of the plurality of flexible electrodes 120 other than the sensing portion 140, and may thus serve as a buffer between the brain and the plurality of flexible electrodes 120. Accordingly, brain damage may be minimized.

The mechanical properties of the polymer layer 130 may vary depending on the temperature. More specifically, the mechanical properties of the polymer layer 130 may decrease as the temperature increases. The polymer layer 130 may have high mechanical properties at room temperature. The polymer layer 130 may have higher mechanical properties than each of the plurality of flexible electrodes 120 at a temperature lower than about 36 °C. For example, the polymer layer 130 may have mechanical properties higher than about 10 Mpa at about 20 °C to about 33 °C. The polymer layer 130 may have mechanical properties of about 3 MPa or less at a temperature of about 36 °C or higher.

The mechanical properties of the polymer layer 130 at a temperature of about 25 °C may be about 30 to about 40 times greater than the mechanical properties of the polymer layer 13 at a temperature of about 36.5 °C. The mechanical properties of the polymer layer 130 at a temperature of about 25 °C may be about 100 to about 110 times greater than the mechanical properties of the polymer layer 13 at a temperature of about 40 °C. The mechanical properties of the polymer layer 130 at a temperature of about 25 °C to about 30 °C may be, for example, about 30 MPa to about 100 MPa. The mechanical properties of the polymer layer at a temperature of about 36 °C to about 40 °C may be, for example, about 0.3 MPa to about 1 MPa.

The polymer layer 130 may have mechanical properties similar to those of the brain at a temperature close to body temperature (e.g., about 36.5 °C), thereby preventing brain damage. In addition, the polymer layer 130 has high mechanical properties at room temperature, and thus a device for measuring brain signals may be easily inserted into the brain.

That is, according to embodiments of the inventive concept, the polymer layer 130 has high mechanical properties at room temperature, and thus a device for measuring brain signals may be easily inserted into the brain. Furthermore, the polymer layer 130 has low mechanical properties at body temperature, and thus brain damage may be prevented. Therefore, the device for measuring brain signals capable of accurately measuring brain signals without brain damage may be provided.

The polymer layer 130 may have a thickness 130D of, for example, about 10 μm to about 100 μm in the first direction D1. The thickness 130D of the polymer layer 130 may indicate a thickness measured along the first direction D1 from the side surface 120S of the plurality of flexible electrodes 120. When the polymer layer 130 has a thickness 130D of less than about 10 μm, sufficient mechanical properties may not be achieved at room temperature. In addition, the polymer layer 130 is too thin to serve as a buffer between the brain and the plurality of flexible electrodes 120. When the polymer layer 130 has a thickness 130D of greater than about 100 μm, the polymer layer 130 may have greater mechanical properties than the brain at a temperature higher than body temperature (e.g., a temperature higher than about 36.5 °C). Accordingly, brain damage may be induced. In addition, the polymer layer 130 may not be uniformly deposited onto the plurality of flexible electrodes 120.

The polymer layer 130 may have a glass transition temperature of, for example, about 34 °C to about 40 °C. The polymer layer 130 may change from crystalline to amorphous at or above the glass transition temperature. The polymer layer 130 may change to amorphous at or above the glass transition temperature, thereby having low mechanical properties.

The polymer layer 130 may include a first repeating unit and a second repeating unit. The first repeating unit may include, for example, an acrylate group (-COO-). The second repeating unit may include, for example, a urethane group (-NH-CO-O-).

The first repeating unit may amount to, for example, about 30 to about 45 parts by weight with respect to 100 parts by weight of the polymer layer, and the second repeating unit may amount to, for example, about 55 to about 70 parts by weight. When the numerical range described above is satisfied, the polymer layer 130 may have a glass transition temperature close to body temperature (about 36.5 °C). In addition, when the numerical range described above is satisfied, the polymer layer 130 may have high mechanical properties at room temperature (e.g., about 25 °C) and have low mechanical properties at a temperature higher than body temperature (about 36.5 °C). More specifically, when the first repeating unit amounts to greater than about 45 parts by weight, the polymer layer 130 may have a glass transition temperature of higher than about 40 °C. When the second repeating unit amounts to less than about 55 parts by weight, the polymer layer 130 may have mechanical properties of higher than about 3 MPa at body temperature.

The first repeating unit may be derived from, for example, stearyl acrylate. The second repeating unit may be derived from, for example, urethane diacrylate.

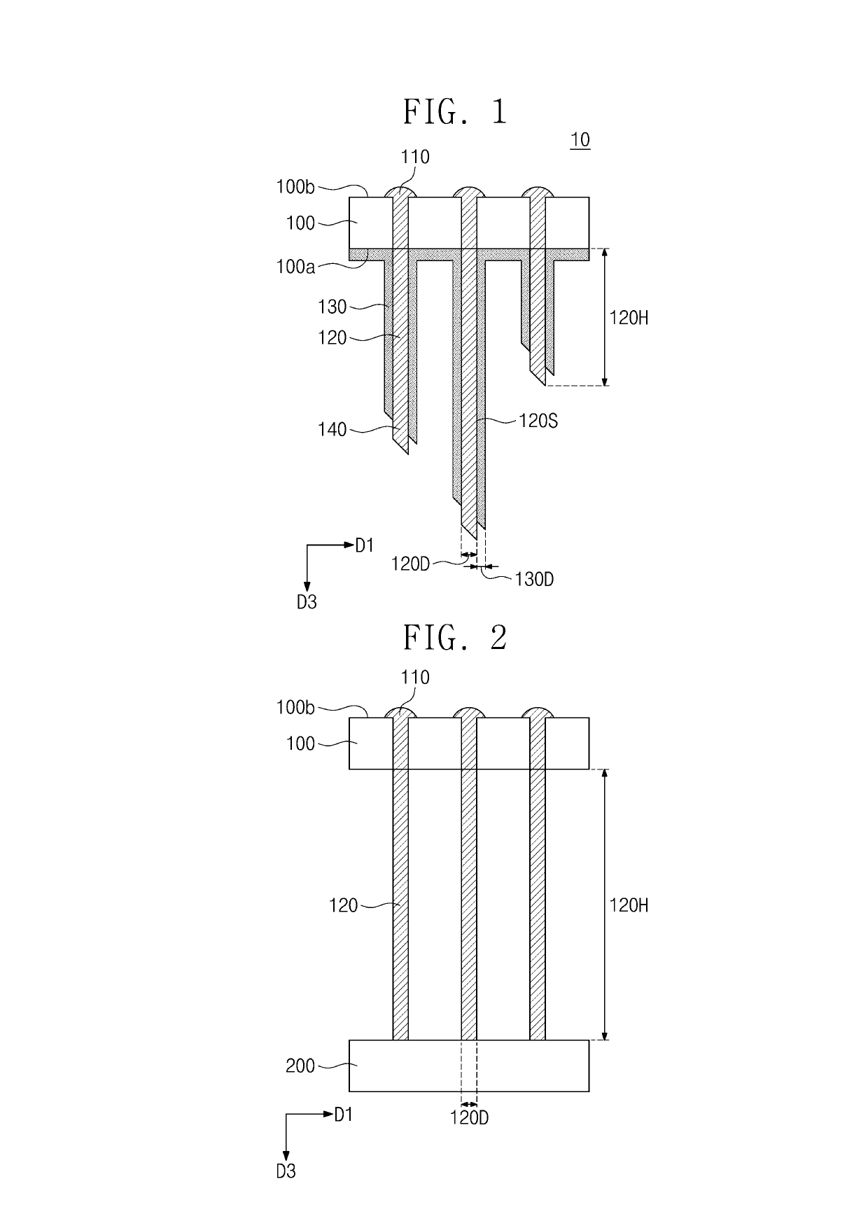

FIGS. 2 to 5 are views showing a method for manufacturing a device for measuring brain signals according to embodiments of the inventive concept.

Referring to FIG. 2, an electrode substrate 100 including a first surface 100a and a second surface 100b opposing each other may be provided. The electrode substrate 100 may include a plurality of electrode connection portions 110 passing through the electrode substrate 100. Each of the plurality of electrode connection portions 110 may be spaced apart in a first direction D1 on the electrode substrate 100.

An end of each of a plurality of flexible electrodes 120 may be mechanically coupled to a corresponding electrode connection portion among the plurality of electrode connection portions 110. Each of the plurality of electrode connection portions 110 may be mechanically coupled to a corresponding flexible electrode among the plurality of flexible electrodes 120. The plurality of flexible electrodes 120 may be spaced apart along the first direction D1. Accordingly, the plurality of flexible electrodes 120 may be fixed to the electrode substrate 100. A length 120H of each of the plurality of flexible electrodes 120 in the third direction D3 of may be the same.

Each of the plurality of flexible electrodes 120 may have mechanical properties of less than about 3 MPa, preferably less than about 1 MPa. Each of the plurality of flexible electrodes 120 may have a thickness 120D of, for example, about 5 μm to about 15 μm. When each of the plurality of flexible electrodes 120 has a thickness 120D of less than about 5 μm, a surface area in contact with the brain may be small, making it difficult to accurately measure brain signals. In addition, when the thickness 120D is greater than about 15 μm, each of the plurality of flexible electrodes 120 may have greater mechanical properties than the brain, causing brain damage.

A dummy substrate 200 may be provided. The other end of each of the plurality of flexible electrodes 120 may be fixed to the dummy substrate 200. The plurality of flexible electrodes 120 may be fixed through the dummy substrate 200 and the electrode substrate 100.

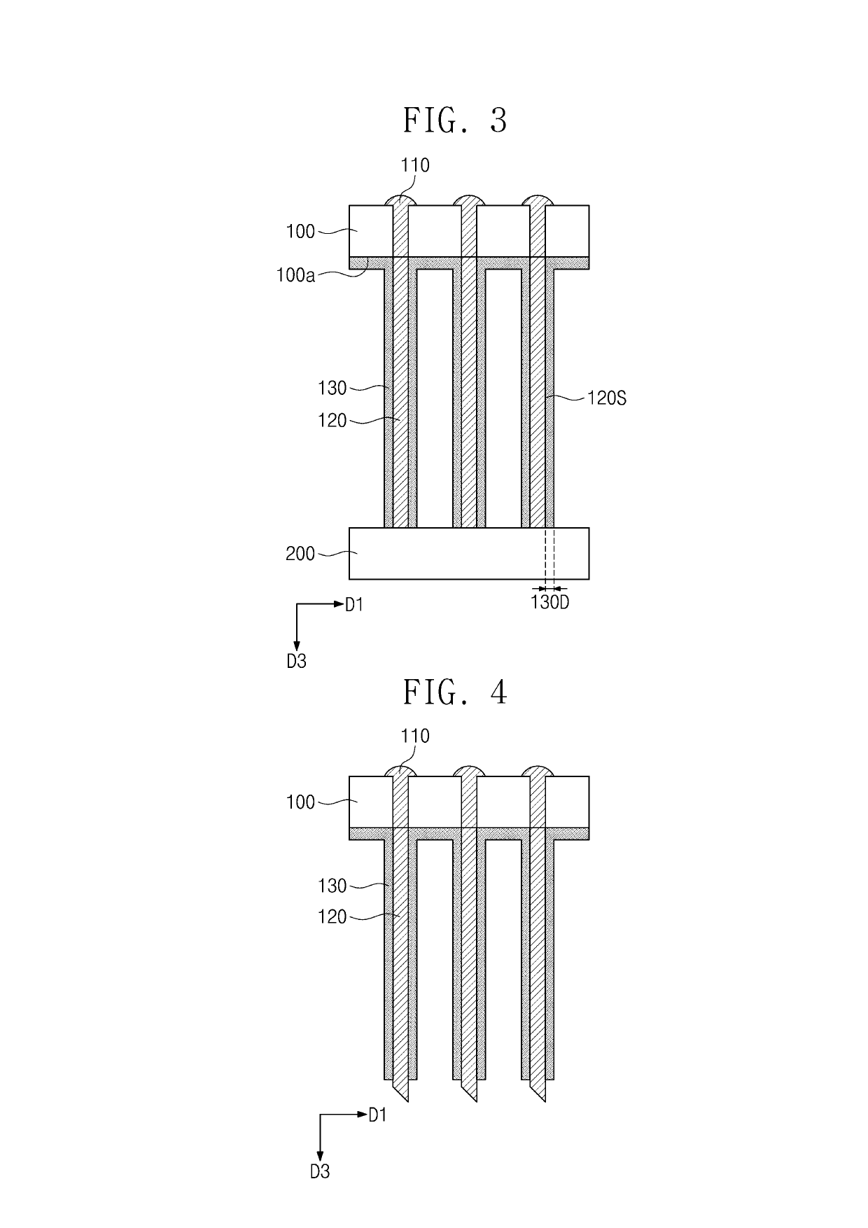

Referring to FIG. 3, a polymer layer 130 may be deposited on a side surface 120S of the plurality of flexible electrodes 120 and the first surface 100a of the electrode substrate. A polymer layer 130 may be uniformly deposited on the side surface 120S of the plurality of flexible electrodes 120. The polymer layer 130 may have a thickness 130D of, for example, about 10 μm to about 100 μm. The thickness 130D of the polymer layer 130 may indicate a thickness measured in the first direction D1 from the side surface 120S of the plurality of flexible electrodes 120.

When the polymer layer 130 has a thickness 130D of less than about 10 μm, sufficient mechanical properties may not be achieved at room temperature. In addition, the polymer layer 130 may not be uniformly deposited onto the plurality of flexible electrodes 120. When the polymer layer 130 has a thickness 130D of greater than about 100 μm, the size of a device for measuring brain signals may become excessively large.

Forming the polymer layer 130 may include, for example, mixing a first compound and a second compound to prepare a first mixture, mixing the first mixture with a crosslinker and a photoinitiator in a solvent to prepare a first solution, and applying light energy to the first solution. Applying light energy may include applying ultraviolet light having a wavelength of about 365 nm.

The first compound may be a compound containing an acrylate group, and the second compound may be a compound containing a multiple bond between carbon atoms, and a urethane group. The multiple bond may be, for example, a double bond (C=C). The first compound may amount to about 35 to about 45 parts by weight with respect to 100 parts by weight of the first mixture. The second compound may amount to about 55 to about 65 parts by weight with respect to 100 parts by weight of the second mixture. The first compound may be, for example, stearyl acrylate. The second compound may be, for example, urethane diacrylate. The crosslinker may amount to about 1 part by weight with respect to 100 parts by weight of the first mixture. The crosslinker may include, for example, TMP-PA.

The photoinitiator may include, for example, DMPA and benzophenone. The DMPA may amount to about 1 part by weight with respect to 100 parts by weight of the first mixture. The benzophenone may amount to 0.5 parts by weight with respect to 100 parts by weight of the first mixture. The solvent may be, for example, any one among ethyl acetate, propylene glycol methyl ether acetate, toluene, xylene, and isopropyl alcohol.

According to another embodiment of the inventive concept, for example, mixing the first compound, the second compound, and the third compound to prepare a second mixture, mixing the second mixture, a crosslinker, and a photoinitiator in a solvent to prepare a second solution, and applying light energy to the second solution. Applying light energy may include applying ultraviolet light having a wavelength of about 365 nm. The first compound may be a compound containing an acrylate group, and the second compound may be a compound containing a multiple bond between carbon atoms, and a urethane group. The third compound may be, for example, a compound having a shorter chain length than the first compound and/or the second compound. The third compound may include, for example, an aryl group. Due to the third compound having a relatively short chain length, the glass crystal temperature may be controlled to a desired temperature, and the mechanical properties may be reduced. The first compound may be, for example, stearyl acrylate. The second compound may be, for example, urethane diacrylate. The third compound may be, for example, tetradecyl acrylate.

Referring to FIG. 4, the dummy substrate 200 may be removed.

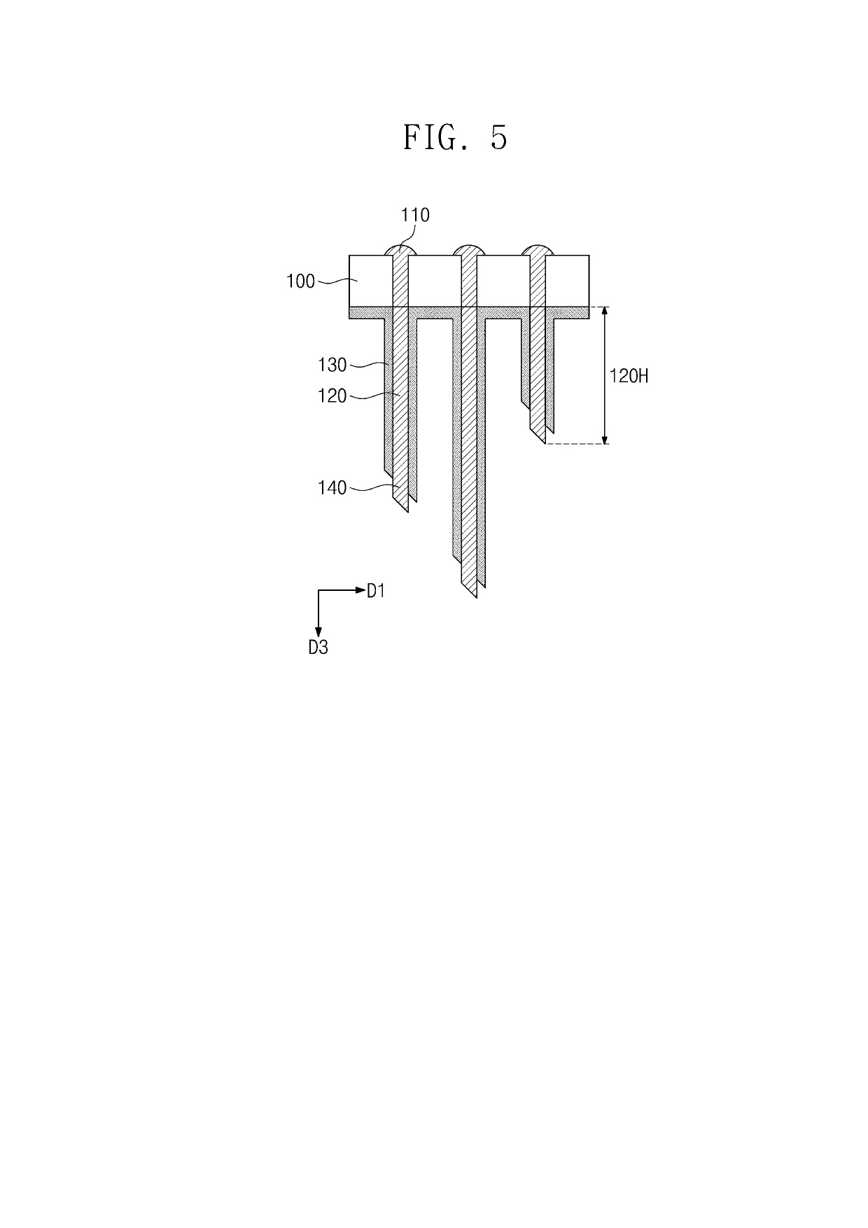

Referring to FIG. 5, the plurality of flexible electrodes 120 may be cut to suit the purpose. The length 120H of the plurality of flexible electrodes 120 in the third direction D3 may be adjusted to suit a target brain region. Accordingly, the length 120H of each of the plurality of flexible electrodes 120 in the third direction D3 may be different. By the cutting process, a sensing portion 140 of each of the plurality of flexible electrodes 120 may be exposed. That is, the polymer layer 130 covering the sensing portion 140 may be removed, thereby exposing the sensing portion 140. Accordingly, the sensing portion may come into contact with the brain.

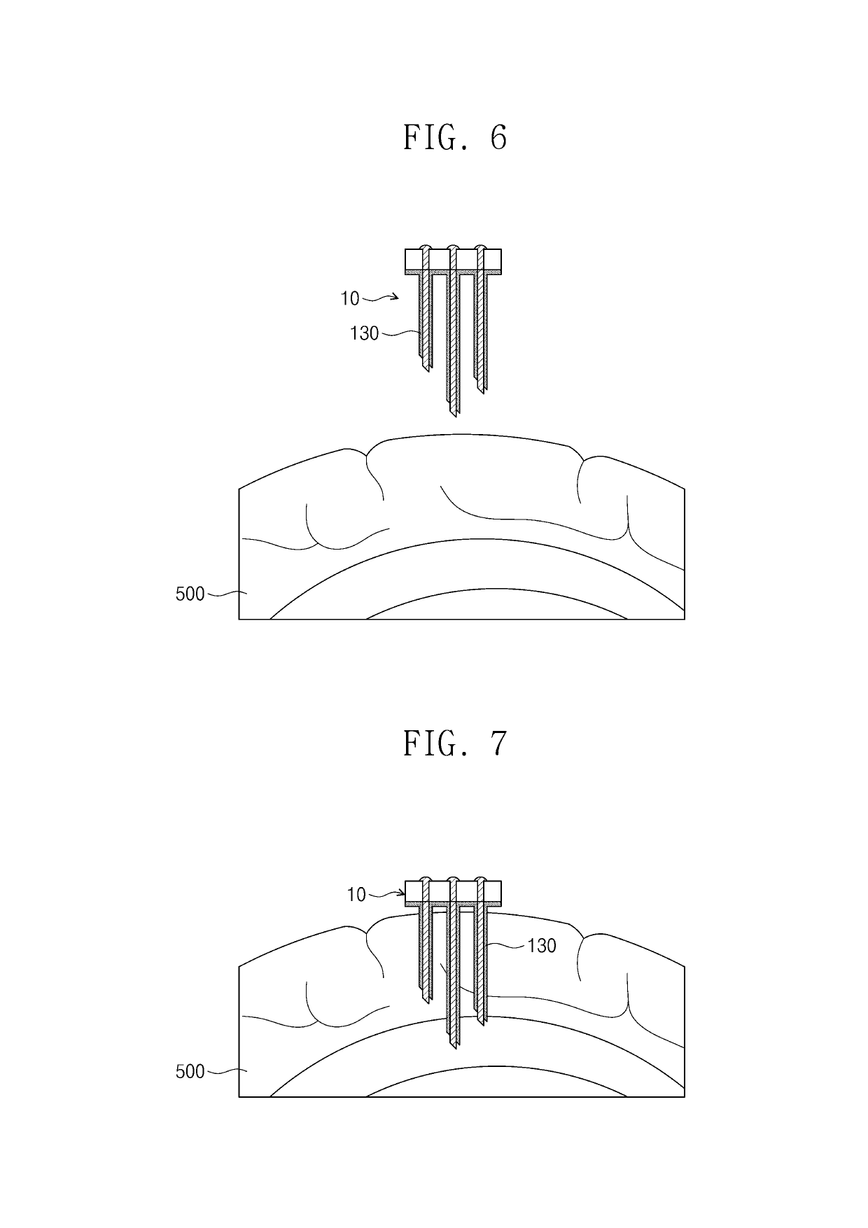

FIGS. 6 and 7 are views showing an insertion process of a device for measuring brain signals according to embodiments of the inventive concept. FIG. 6 is a view showing a state before a device for measuring brain signals according to embodiments of the inventive concept is inserted into the brain. FIG. 7 is a view showing a state before a device for measuring brain signals according to embodiments of the inventive concept is inserted into the brain. For simplicity of description, descriptions overlapping those of the device for measuring brain signals described with reference to FIG. 1 will be omitted.

Referring to FIG. 6, the polymer layer 130 may have high mechanical properties at room temperature. The polymer layer 130 has high mechanical properties at room temperature, and thus the device for measuring brain signals 10 may be easily inserted into brain 500. The polymer layer 130 and the device for measuring brain signals 10 may be substantially the same as the polymer layer 130 and the device for measuring brain signals 10 of FIG. 1.

Referring to FIG. 7, the polymer layer 130 may have low mechanical properties at or above body temperature. The polymer layer 130 and the device for measuring brain signals may be substantially the same as the polymer layer 130 and the device for measuring brain signals 10 of FIG. 1.

The polymer layer 130 has low stiffness at body temperature, and thus damage to the brain 500 may be minimized. The polymer layer 130 has mechanical properties similar to those of the brain at or above body temperature, and thus the shape of the polymer layer 130 may be deformed according to the movement of the brain 500. Accordingly, the polymer layer 130 may serve as a buffer between the brain and the plurality of flexible electrodes 120, thereby minimizing damage to the brain 500.

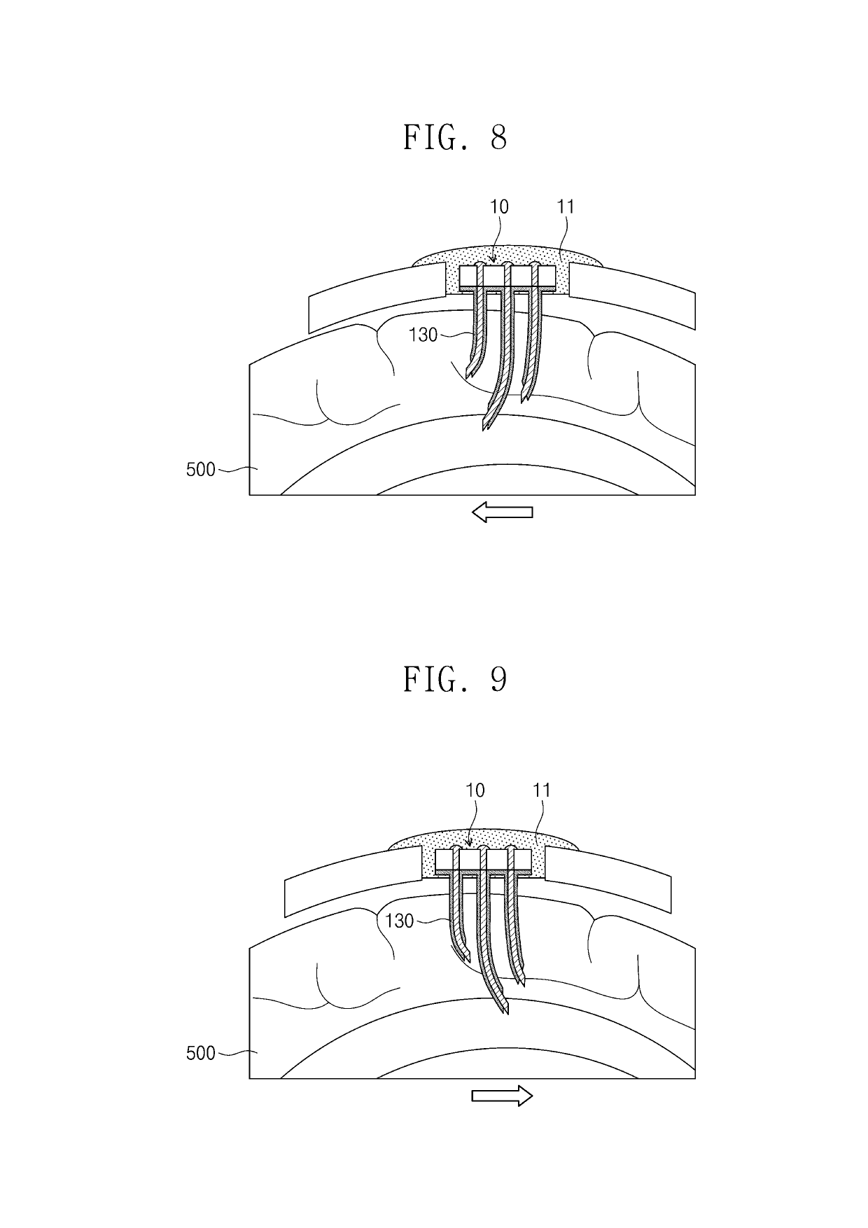

FIGS. 8 and 9 are views showing a state in which a device for measuring brain signals according to embodiments of the inventive concept is inserted into a brain. For simplicity of description, descriptions overlapping those of the device for measuring brain signals described with reference to FIG. 1 will be omitted.

Referring to FIG. 8, when the brain 500 moves to the left, the device for measuring brain signals 10 may also move to the left. The polymer layer 130 and the device for measuring brain signals 10 may be substantially the same as the polymer layer 130 and the brain signal device of FIG. 1. Furthermore, the device for measuring brain signals 10 may be fixed by a fixing device 11.

The brain 500 may be immersed in cerebrospinal fluid and fluidly movable. The polymer layer 130 of the device for measuring brain signals 10 may have low mechanical properties at a high temperature (e.g., a temperature higher than body temperature), thereby preventing brain damage.

That is, the polymer layer 130 has mechanical properties similar to those of the brain at or above body temperature, and thus the shape of the polymer layer 130 may be deformed according to the movement of the brain. Accordingly, the polymer layer 130 may serve as a buffer between the brain and the plurality of flexible electrodes 120, thereby minimizing damage to the brain.

Referring to FIG. 9, when the brain 500 moves to the right, the device for measuring brain signals 10 may also move to the right. The polymer layer 130 and the device for measuring brain signals 10 may be substantially the same as the polymer layer 130 and the brain signal device of FIG. 1.

The device for measuring brain signals 10 may also move in accordance with the movement direction of the brain 500, thereby measuring brain signals while minimizing brain damage. Furthermore, the device for measuring brain signals may be fixed by a fixing device 11.

The polymer layer 130 according to embodiments of the inventive concept has low mechanical properties at a temperature higher than body temperature, and may thus move in accordance with the movement of the brain. Accordingly, brain signals may be continuously measured without causing damage to the brain.

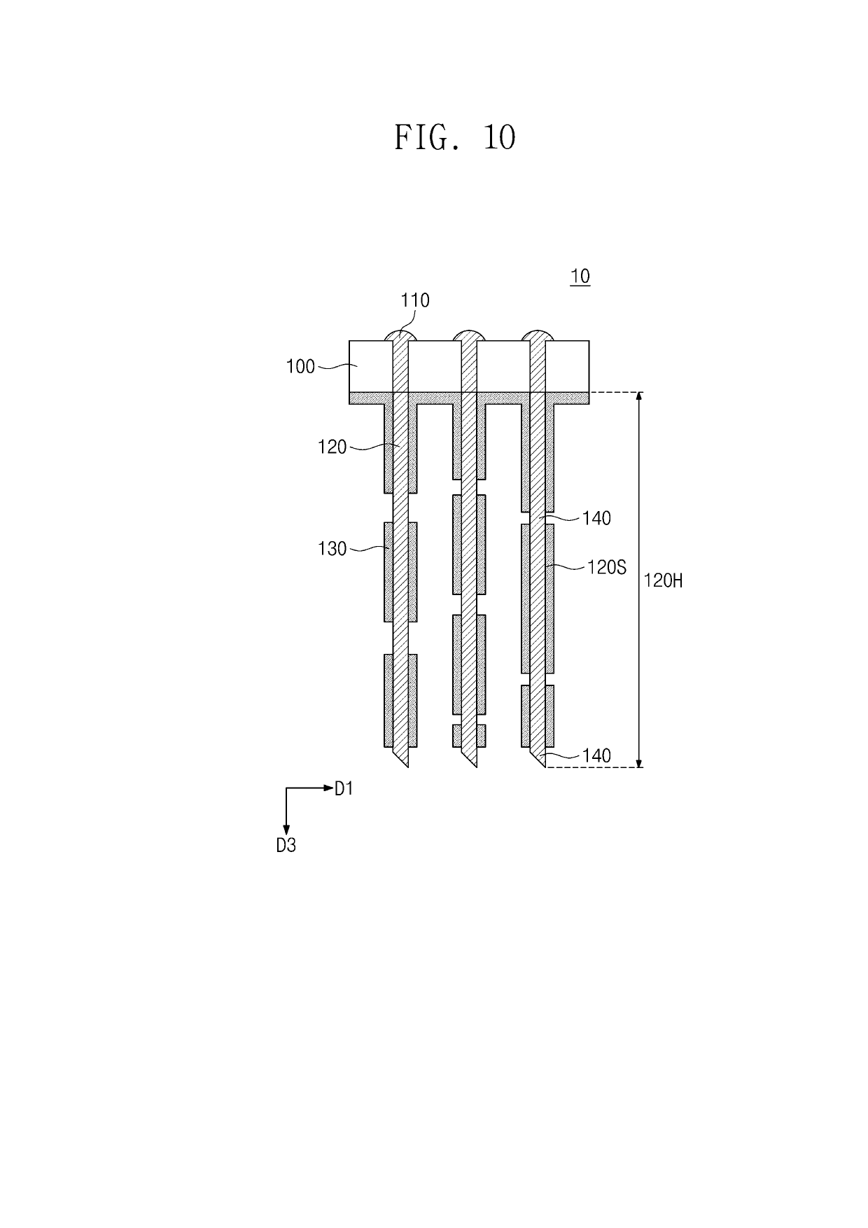

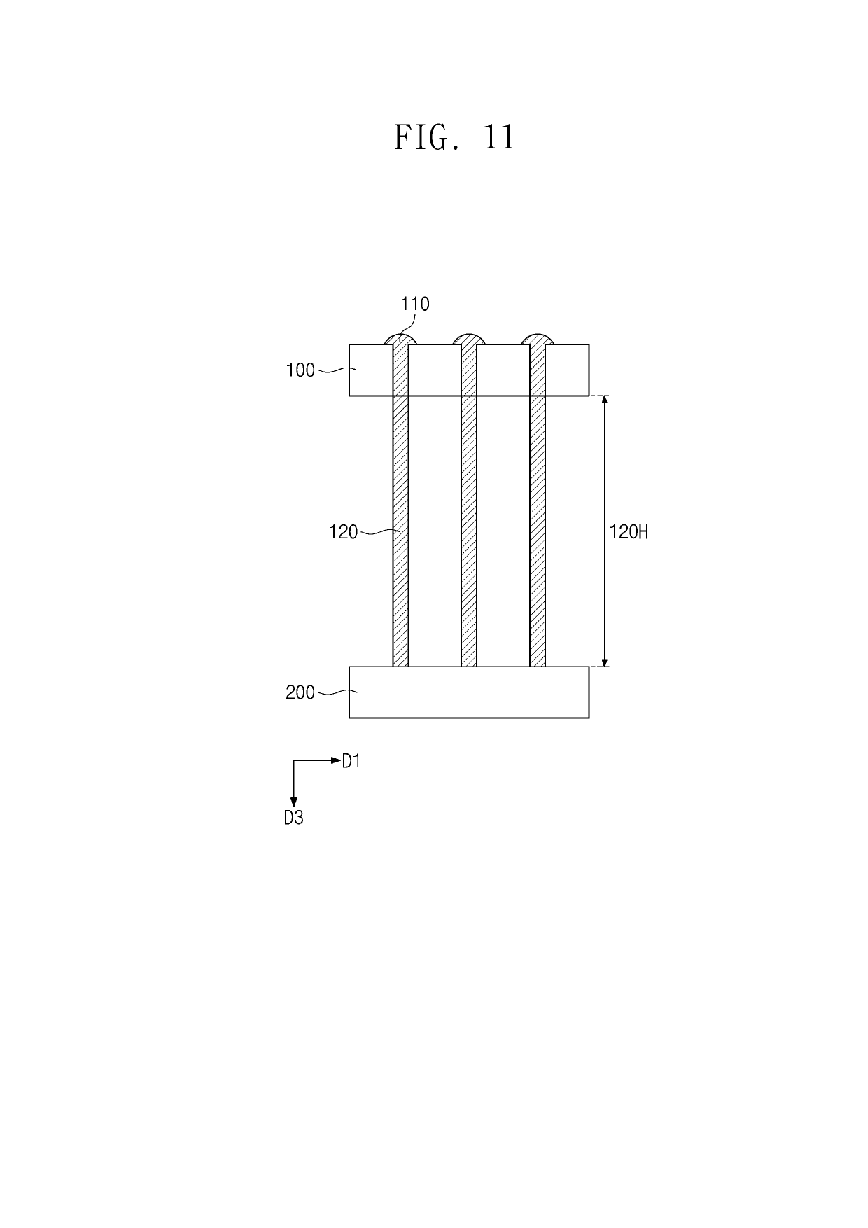

FIG. 10 is a view showing a device for measuring brain signals according to some embodiments of the inventive concept. For simplicity of description, descriptions overlapping those of the device for measuring brain signals described with reference to FIG. 1 will be omitted.

The device for measuring brain signals 10 may include an electrode substrate 100, a plurality of flexible electrodes 120 on the electrode substrate 100, and a polymer layer 130 covering the plurality of flexible electrodes 120. The electrode substrate 100 may include a plurality of electrode connection portions 110 passing through the electrode substrate 100. The plurality of electrode connection portions 110 may be spaced apart in a first direction D1.

The plurality of flexible electrodes 120 may be spaced apart in the first direction D1. A length 120H of each of the plurality of flexible electrodes 120 in the third direction D3 may be the same. In FIG. 10, three flexible electrodes 120 are shown, but the inventive concept is not limited thereto, and more than three flexible electrodes 120 may be provided.

The plurality of flexible electrodes 120 may include, for example, a conductive material. Each of the plurality of flexible electrodes 120 may have mechanical properties of less than about 3 MPa, preferably less than about 1 MPa. More specifically, each of the plurality of flexible electrodes 120 may have mechanical properties of, for example, less than about 1 MPa at a temperature of about 25 °C.

Each of the plurality of flexible electrodes 120 may include a plurality of sensing portion 140. The plurality of sensing portions 140 may be spaced apart along the third direction D3. Accordingly, signals occurring in various regions of the brain may be measured simultaneously.

The polymer layer 130 may cover at least a portion of a side surface 120S of each of the plurality of flexible electrodes 120. The polymer layer 130 covers the plurality of flexible electrodes 120, and may thus minimize direct contact between the brain and a metal.

The polymer layer 130 may expose the plurality of sensing portion 140 of each of the plurality of flexible electrodes 120. Accordingly, the plurality of sensing portions 140 may come into contact with the brain. The polymer layer 130 may cover a side surface 120S of each of the plurality of flexible electrodes 120 other than the plurality of sensing portions 140, and may thus minimize damage to the brain by the plurality of flexible electrodes 120.

That is, according to embodiments of the inventive concept, the polymer layer 130 has high mechanical properties at room temperature, and thus the device for measuring brain signals 10 may be easily inserted into the brain. Furthermore, the polymer layer 130 has low mechanical properties at body temperature, and thus brain damage may be prevented. Therefore, the device for measuring brain signals 10 capable of accurately measuring brain signals without brain damage may be provided.

The other structure may be substantially the same as the device for measuring brain signals described with reference to FIG. 1.

FIGS. 11 to 14 are views showing a method for manufacturing a device for measuring brain signals according to embodiments of the inventive concept. For simplicity of description, descriptions overlapping those of the device for measuring brain signals described with reference to FIG. 1 will be omitted.

Referring to FIG. 11, an electrode substrate 100 may be provided. The electrode substrate 100 may include a plurality of electrode connection portions 110 passing through the electrode substrate 100.

An end of each of a plurality of flexible electrodes 120 may be mechanically coupled to a corresponding electrode connection portion among the plurality of electrode connection portions 110. A length 120H of each of the plurality of flexible electrodes 120 in the third direction D3 may be the same. Accordingly, the plurality of flexible electrodes 120 may be fixed to the electrode substrate 100. A dummy substrate 200 may be provided. The other end of each of the plurality of flexible electrodes 120 may be fixed to the dummy substrate 200.

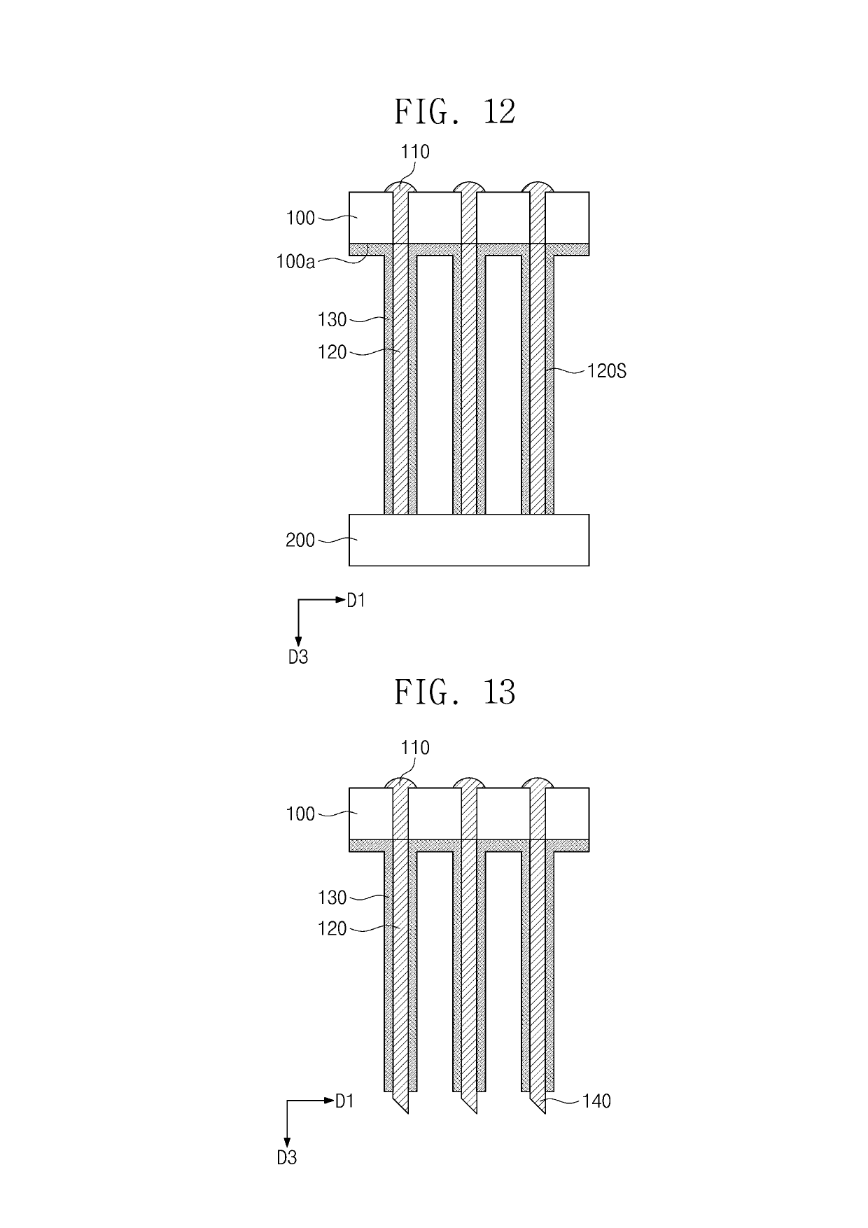

Referring to FIG. 12, a polymer layer 130 may be deposited on a side surface of the plurality of flexible electrodes 120 and a first surface 100a of the electrode substrate. The polymer layer 130 may be uniformly deposited on a side surface 120S of the plurality of flexible electrodes 120. Forming the polymer layer 130 may include, for example, a photocuring reaction of a first repeating unit and a second repeating unit. The first repeating unit may include an acrylate group (-COO-). The second repeating unit may include, for example, a multiple bond between carbon atoms, and may include, for example, a double bond (C=C).

Referring to FIG. 13, a dummy substrate (see 200 of FIG. 12) may be removed.

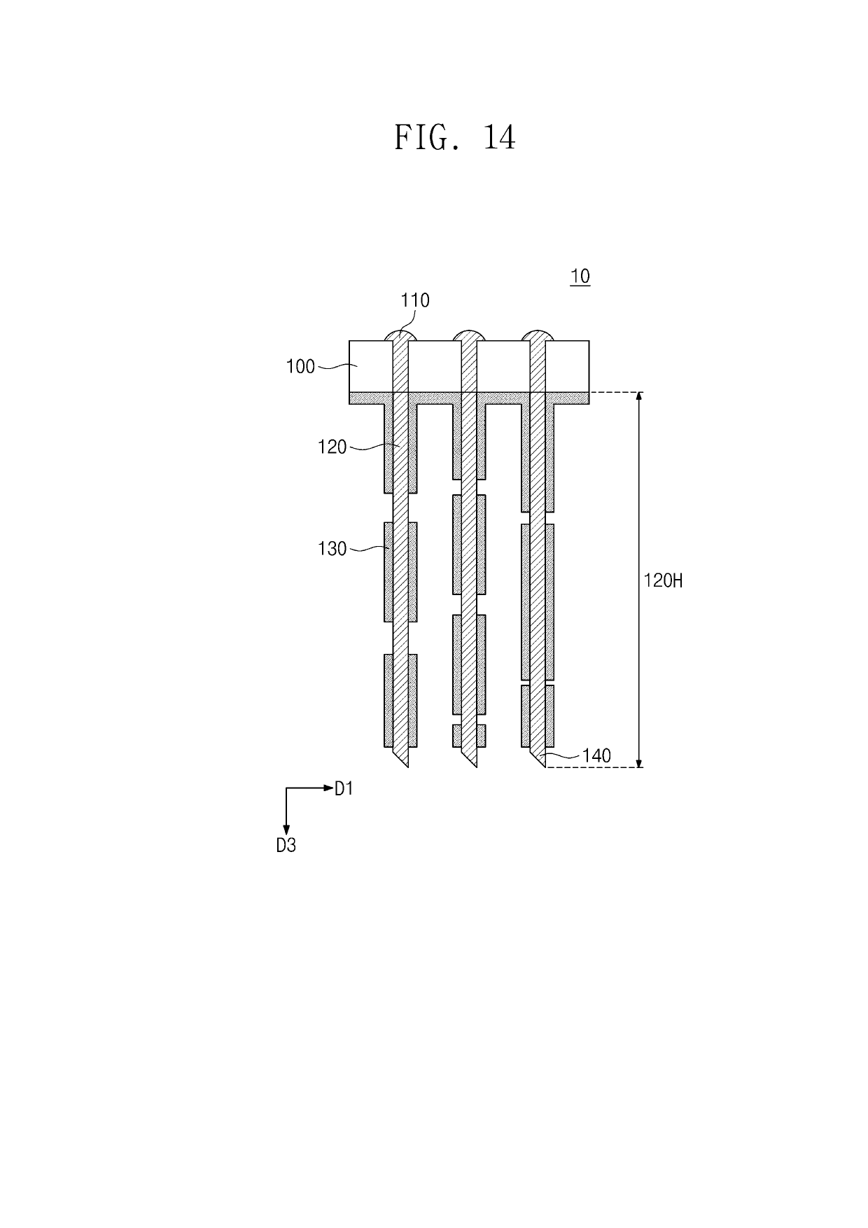

Referring to FIG. 14, the polymer layer 130 covering the plurality of sensing portions 140 of each of the plurality of flexible electrodes 120 may be removed. Accordingly, the plurality of sensing portions 140 may be exposed. That is, the plurality of sensing portions 140 spaced apart in the third direction D3 are exposed, and thus various brain regions may be measured. Referring to FIG. 14, the cutting process described with reference to FIG. 5 may be omitted. Accordingly, the length 120H of each of the plurality of flexible electrodes 120 in the third direction D3 may be the same.

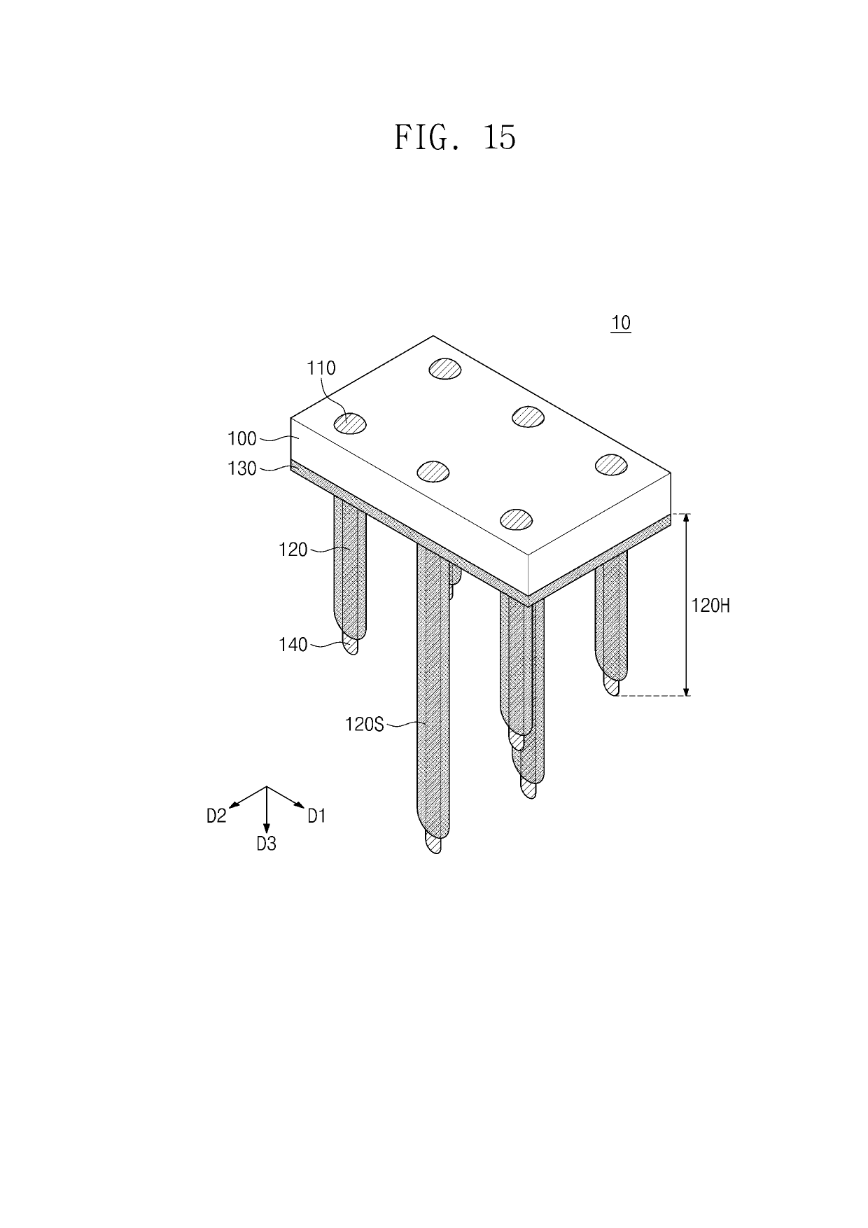

FIG. 15 is a view showing a device for measuring brain signals according to some embodiments of the inventive concept. For simplicity of description, descriptions overlapping those of the device for measuring brain signals described with reference to FIG. 1 will be omitted.

A device for measuring brain signals may include an electrode substrate 100, a plurality of flexible electrodes 120 on the electrode substrate 100, and a polymer layer 130 covering the plurality of flexible electrodes 120. The electrode substrate 100 may include a plurality of electrode connection portions 110 passing through the electrode substrate 100.

The plurality of electrode connection portions 110 may be spaced apart in a first direction D1 and a second direction D2. The second direction D2 may be a direction parallel to a first surface 100a of the electrode substrate 100 and crossing the first direction D1.

The plurality of flexible electrodes 120 may be spaced apart in the first direction D1 and the second direction D2. The arrangement of the plurality of flexible electrodes 120 may not be limited to FIG. 15. The plurality of flexible electrodes 120 may be arranged irregularly and may be arranged according to target brain measurement regions. In addition, in FIG. 15, six flexible electrodes 120 are shown, but the inventive concept is not limited thereto, and more than six flexible electrodes 120 may be provided.

Each of the plurality of flexible electrodes 120 may extend in the third direction D3. A length 120H of each of the plurality of flexible electrodes 120 in a third direction D3 may be different. Accordingly, a sensing portion 140, which will be described later, may be disposed at various locations. Therefore, signals occurring in various regions of the brain may be measured simultaneously. In FIG. 15, six flexible electrodes 120 are shown, but the inventive concept is not limited thereto, and more than six flexible electrodes 120 may be provided.

The plurality of flexible electrodes 120 may include, for example, a conductive material. Each of the plurality of flexible electrodes 120 may have mechanical properties of less than about 3 MPa, preferably less than about 1 MPa.

Each of the plurality of flexible electrodes 120 may include a sensing portion 140. The sensing portion 140 may come into contact with the brain and measure signals occurring in the brain. The sensing portion 140 may be disposed, for example, at an end of each of the plurality of flexible electrodes 120, but is not limited thereto. A length 120H of each of the plurality of flexible electrodes 120 in the third direction D3 is different, and thus the sensing portion 140 may be provided at various locations. Accordingly, signals occurring in various regions of the brain may be measured simultaneously.

The polymer layer 130 may cover at least a portion of a side surface 120S of each of the plurality of flexible electrodes 120. The polymer layer 130 covers the plurality of flexible electrodes 120, and may thus minimize direct contact between the brain and an electrode. The polymer layer 130 may expose the sensing portion 140 of each of the plurality of flexible electrodes 120. Accordingly, the sensing portion 140 may come into contact with the brain. That is, the polymer layer 130 may cover a side surface 120S of each of the plurality of flexible electrodes 120 other than the sensing portion 140, and may thus minimize damage to the brain by the plurality of flexible electrodes 120.

The other structure may be substantially the same as the device for measuring brain signals described with reference to FIG. 1.

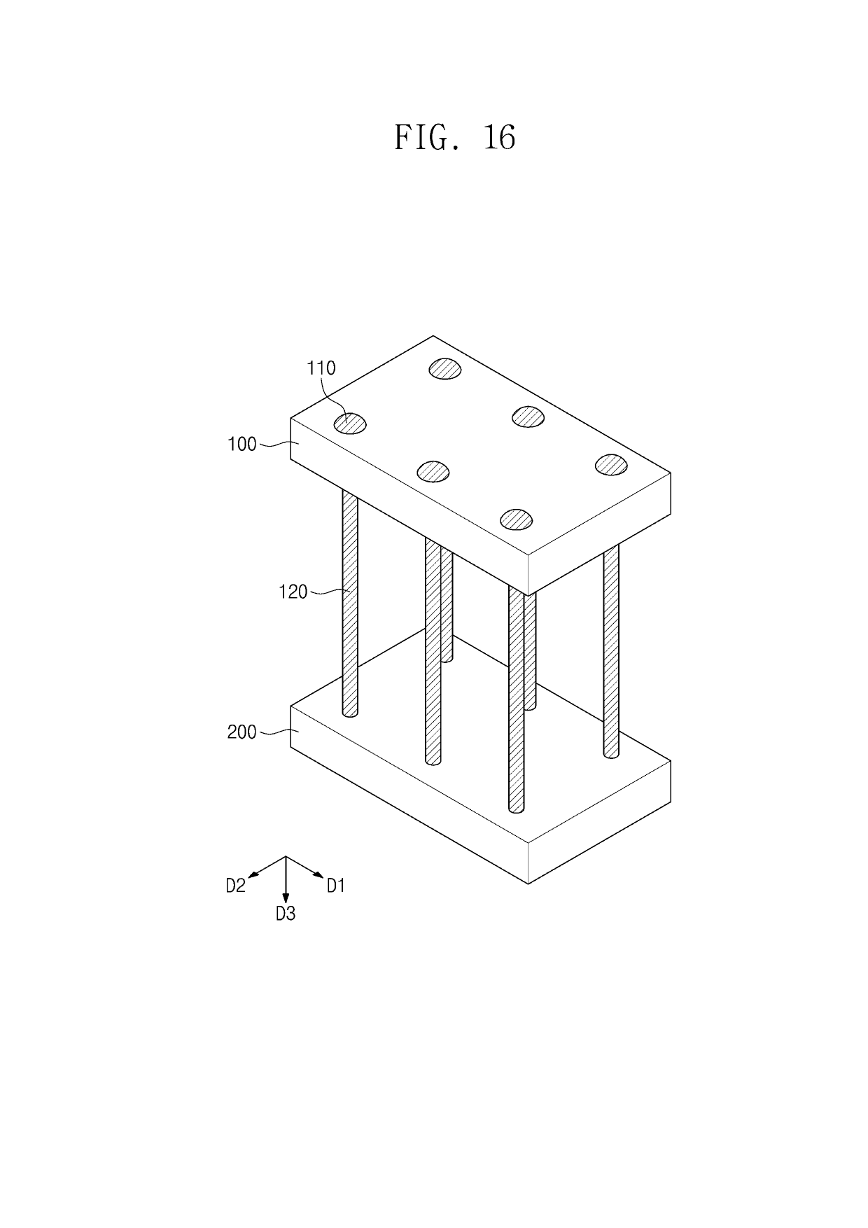

FIGS. 16 to 19 are views showing a method for manufacturing a device for measuring brain signals according to embodiments of the inventive concept. For simplicity of description, descriptions overlapping those of the device for measuring brain signals described with reference to FIG. 1 will be omitted.

Referring to FIG. 16, an electrode substrate 100 may be provided. The electrode substrate 100 may include a plurality of electrode connection portions 110 passing through the electrode substrate 100. The plurality of electrode connection portions 110 may be spaced apart in a first direction D1 and a second direction D2.

An end of each of a plurality of flexible electrodes 120 may be mechanically coupled to a corresponding electrode connection portion among the plurality of electrode connection portions 110. Accordingly, the plurality of flexible electrodes 120 may be fixed to the electrode substrate 100. A length 120H of each of the plurality of flexible electrodes 120 in a third direction D3 of may be the same. A dummy substrate 200 may be provided. The other end of each of the plurality of flexible electrodes 120 may be fixed to the dummy substrate 200.

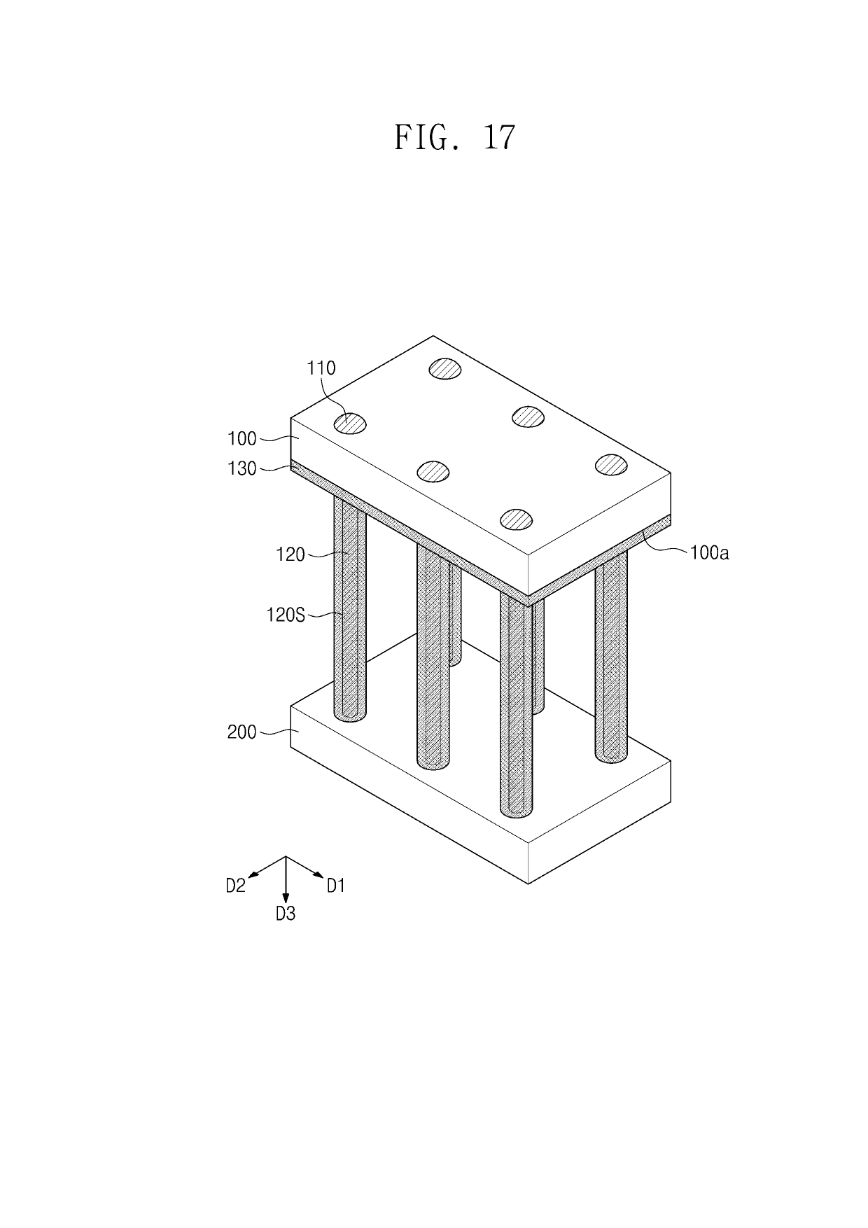

Referring to FIG. 17, a polymer layer 130 may be deposited on a side surface 120S of the plurality of flexible electrodes 120 and a first surface 100a of the electrode substrate. The polymer layer 130 may be uniformly deposited on a side surface of the plurality of flexible electrodes 120. Forming the polymer layer 130 may include, for example, a photocuring reaction of a first repeating unit and a second repeating unit. A first repeating unit may include an acrylate group (-COO-). A second repeating unit may include, for example, a multiple bond between carbon atoms, and may include, for example, a double bond (C=C).

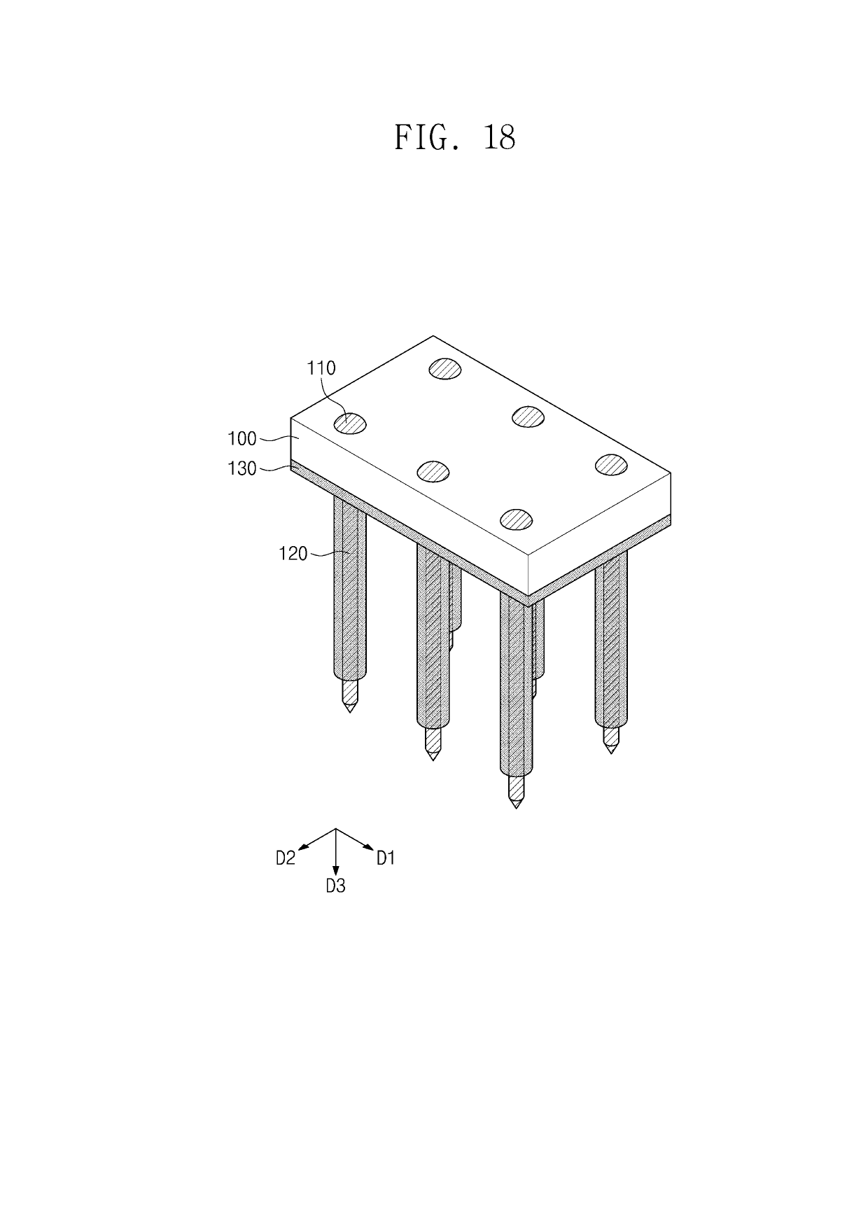

Referring to FIG. 18, a dummy substrate 200 may be removed.

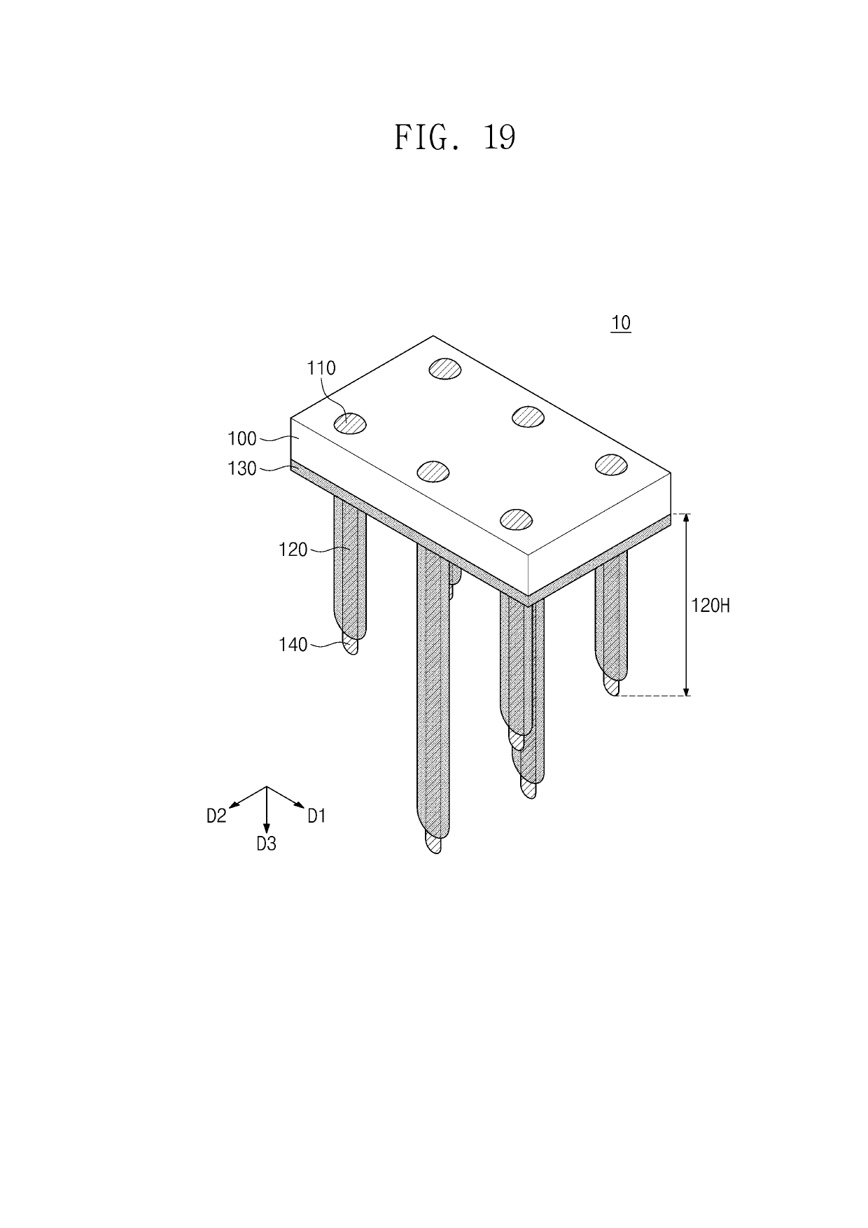

Referring to FIG. 19, the plurality of flexible electrodes 120 may be cut to suit the purpose. That is, a length 120H of the plurality of flexible electrodes 120 in the third direction D3 may be adjusted to suit a target brain region. Accordingly, the length 120H of each of the plurality of flexible electrodes 120 in the third direction D3 may be different. By the cutting process, a sensing portion 140 of each of the plurality of flexible electrodes 120 may be exposed. That is, the polymer layer 130 covering the sensing portion 140 may be removed, thereby exposing the sensing portion 140.

That is, according to some embodiments of the inventive concept, the plurality of flexible electrodes 120 may be disposed three-dimensionally. Furthermore, the length 120H of each of the plurality of flexible electrodes 120 in the third direction D3 is different, and thus the sensing portion 140 may be disposed at various locations. Accordingly, various brain regions may be measured simultaneously.

The other structure may be substantially the same as the device for measuring brain signals described with reference to FIG. 1.

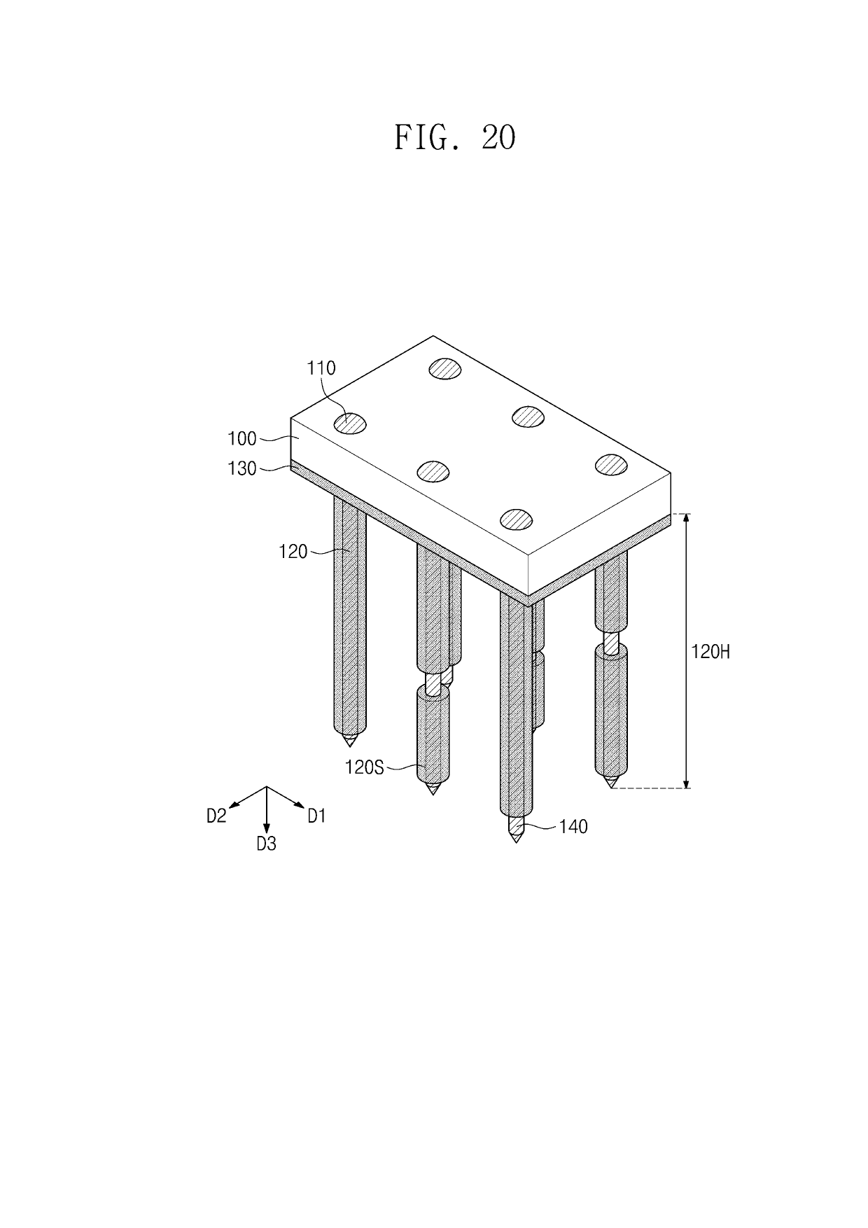

FIG. 20 is a view showing a device for measuring brain signals according to some embodiments of the inventive concept. For simplicity of description, descriptions overlapping those of the device for measuring brain signals described with reference to FIG. 1 will be omitted.

Referring to FIG. 20, a device for measuring brain signals 10 may include an electrode substrate 100, a plurality of flexible electrodes 120 on the electrode substrate 100, and a polymer layer 130 covering the plurality of flexible electrodes 120.

The electrode substrate 100 may include a plurality of electrode connection portions 110 passing through the electrode substrate 100. The plurality of electrode connection portions 110 may be spaced apart in a first direction D1 and a second direction D2. The second direction D2 may be a direction parallel to a first surface 100a of the electrode substrate 100 and crossing the first direction D1.

The plurality of flexible electrodes 120 may be spaced apart in the first direction D1 and the second direction D2. That is, the plurality of flexible electrodes 120 may be disposed three-dimensionally with respect to each other. The arrangement of the plurality of flexible electrodes 120 may not be limited to FIG. 20. The plurality of flexible electrodes 120 may be arranged irregularly and may be arranged according to target brain measurement regions. In addition, in FIG. 20, six flexible electrodes 120 are shown, but the inventive concept is not limited thereto, and more than six flexible electrodes 120 may be provided.

Each of the plurality of flexible electrodes 120 may extend in a third direction D3. A length 120H of each of the plurality of flexible electrodes 120 in the third direction D3 may be the same. The plurality of flexible electrodes 120 may include, for example, a conductive material. Each of the plurality of flexible electrodes 120 may have mechanical properties of less than about 3 MPa, preferably less than about 1 MPa.

Each of the plurality of flexible electrodes 120 may include a plurality of sensing portions 140. The plurality of sensing portions 140 may be spaced apart in the third direction D3. Accordingly, signals occurring in various regions of the brain may be measured simultaneously.

The polymer layer 130 may cover at least a portion of a side surface 120S of each of the plurality of flexible electrodes 120. The polymer layer 130 may cover the plurality of flexible electrodes, thereby minimizing direct contact between the brain and an electrode.

The polymer layer 130 may expose the plurality of sensing portions 140 of each of the plurality of flexible electrodes 120. Accordingly, the plurality of sensing portions 140 may come into contact with the brain. That is, the polymer layer 130 may cover a side surface of each of the plurality of flexible electrodes 120 other than the plurality of sensing portions 140, and may thus minimize damage to the brain by the plurality of flexible electrodes 120.

The other structure may be substantially the same as the device for measuring brain signals described with reference to FIG. 1.

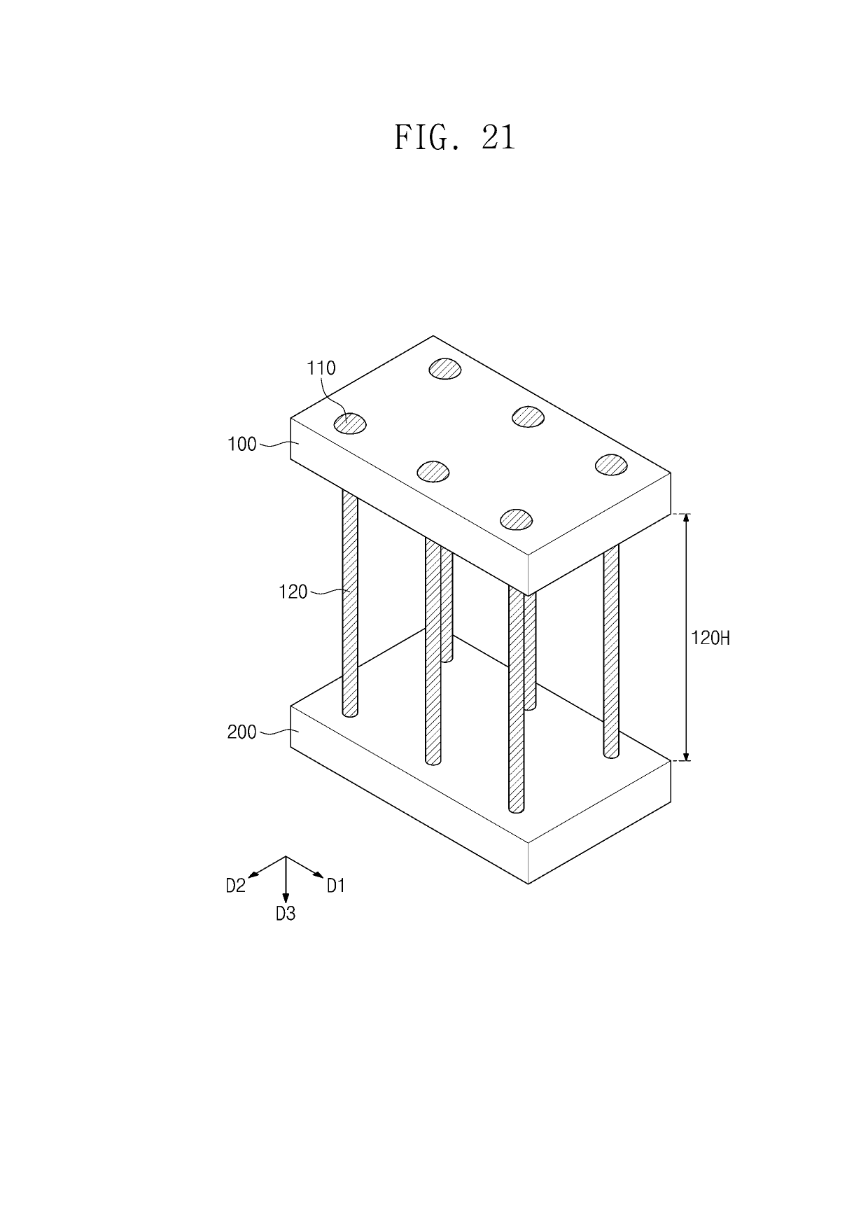

FIGS. 21 to 24 are views showing a method for manufacturing a device for measuring brain signals according to embodiments of the inventive concept.

Referring to FIG. 21, an electrode substrate 100 may be provided. The electrode substrate 100 may include a plurality of electrode connection portions 110 passing through the electrode substrate 100. That is, the plurality of electrode connection portions 110 may be spaced apart in a first direction D1 and a second direction D2.

An end of each of a plurality of flexible electrodes 120 may be mechanically coupled to a corresponding electrode connection portion among the plurality of electrode connection portions 110. Accordingly, the plurality of flexible electrodes 120 may be fixed to the electrode substrate 100. A dummy substrate 200 may be provided. The other end of each of the plurality of flexible electrodes 120 may be fixed to the dummy substrate 200.

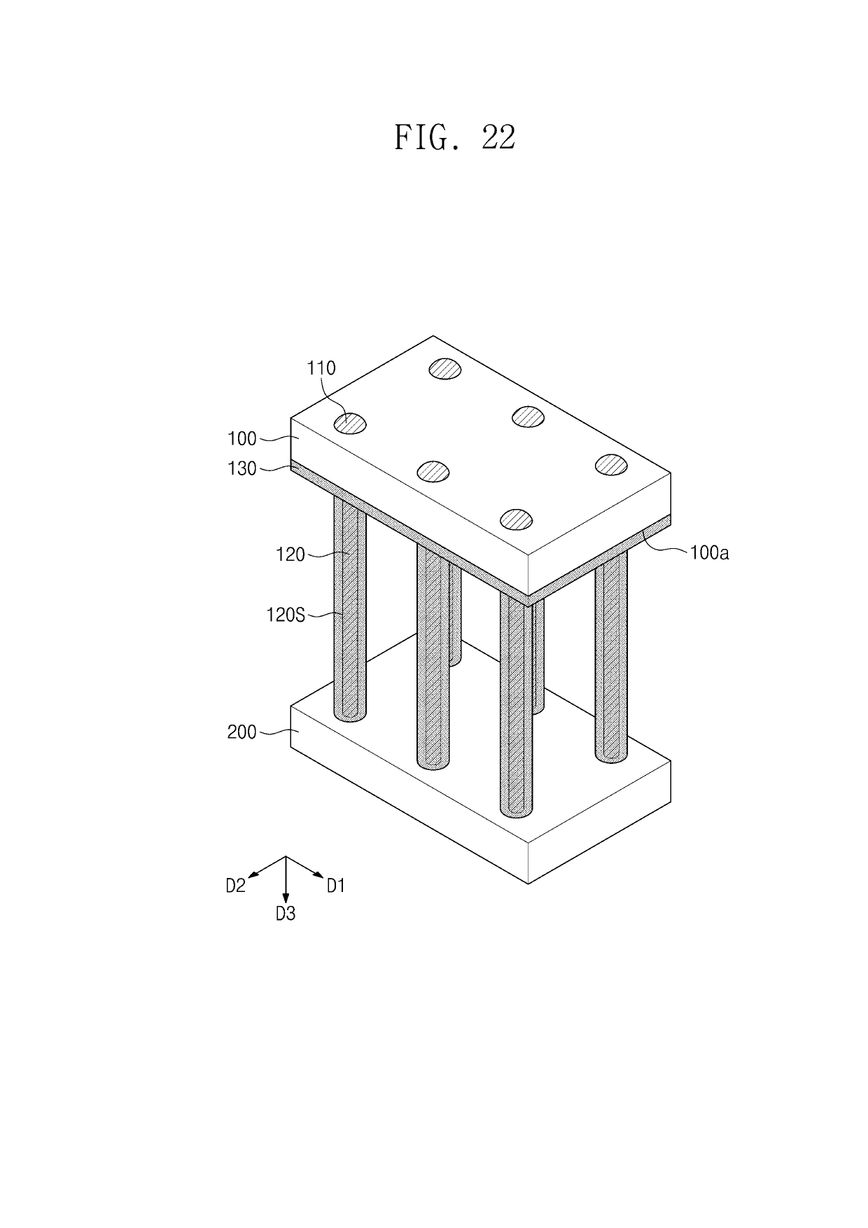

Referring to FIG. 22, a polymer layer 130 may be deposited on a side surface of the plurality of flexible electrodes 120 and a first surface 100a of the electrode substrate. The polymer layer 130 may be uniformly deposited on a side surface of the plurality of flexible electrodes 120.

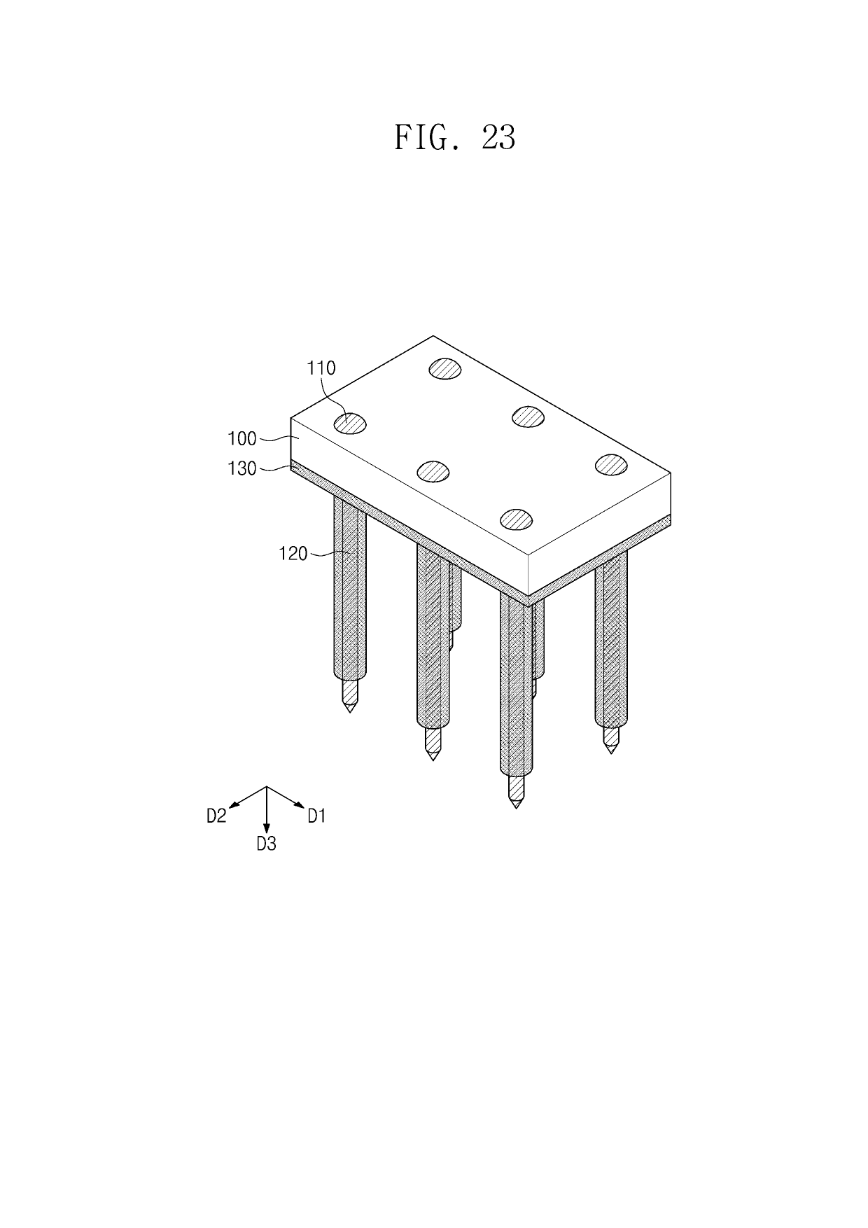

Referring to FIG. 23, a dummy substrate 200 may be removed.

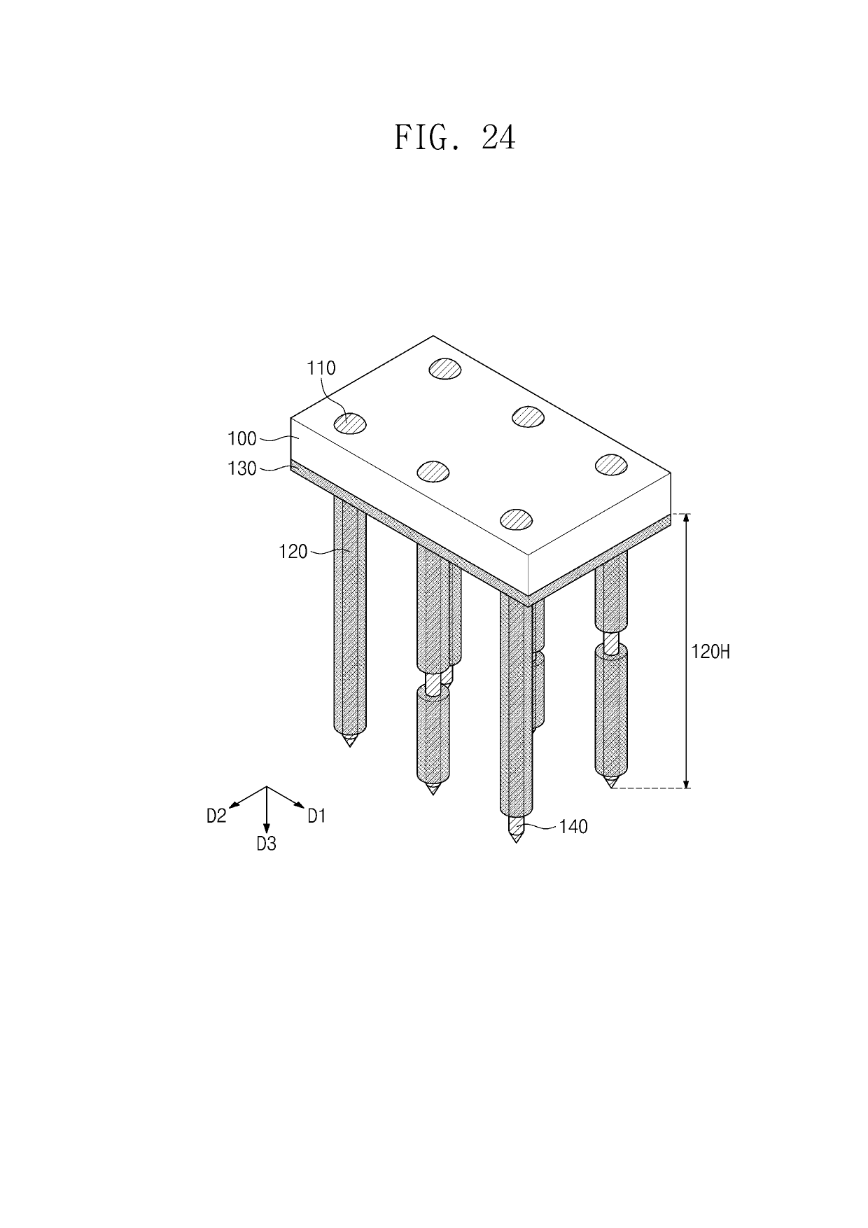

Referring to FIG. 24, the polymer layer 130 covering each of the plurality of sensing portions 140 of the plurality of flexible electrodes 120 may be removed. Accordingly, the plurality of sensing portions 140 may be exposed. That is, the plurality of sensing portions 140 spaced apart in the third direction D3 are exposed, and thus various brain regions may be measured. Referring to FIG. 24, the cutting process of FIG. 19 may be omitted. Accordingly, the length 120H of each of the plurality of flexible electrodes 120 in the third direction D3 may be the same.

The other structure may be substantially the same as the device for measuring brain signals described with reference to FIG. 1.

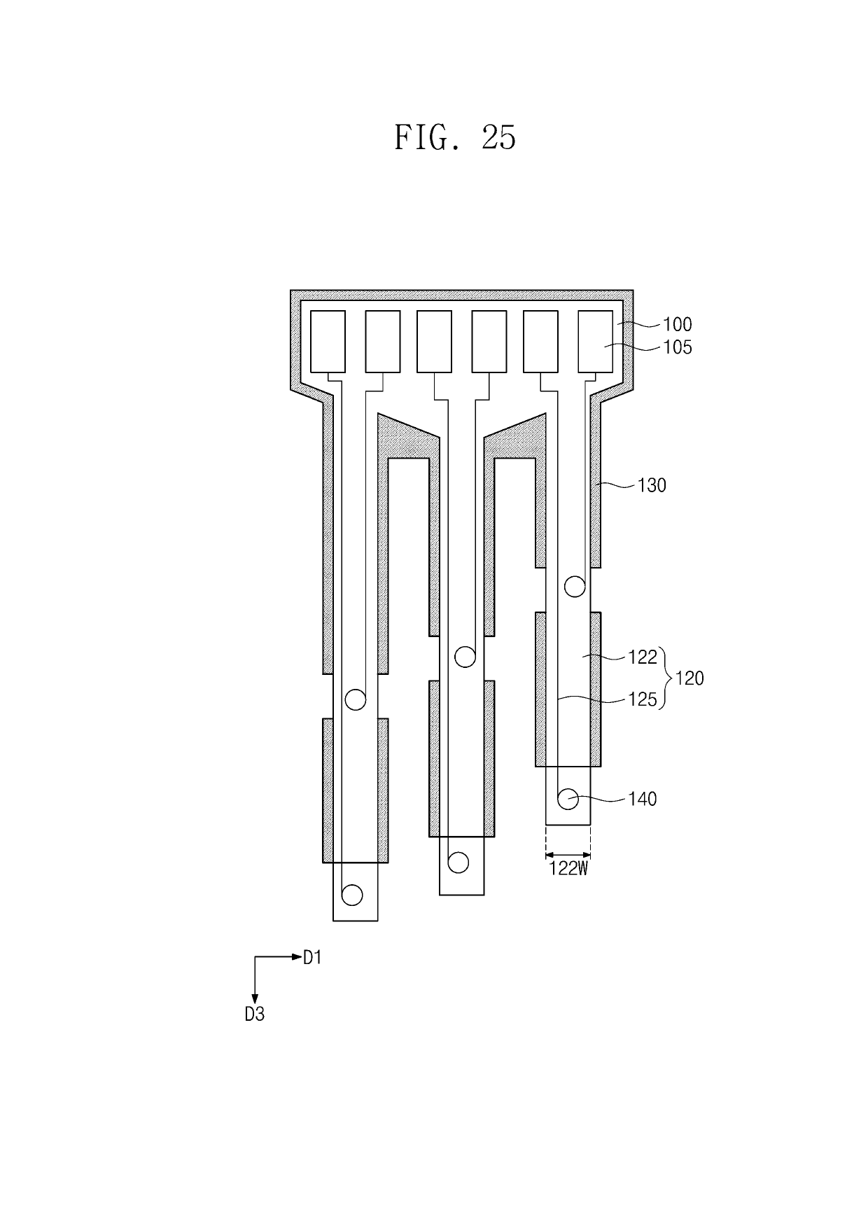

FIG. 25 is a cross-sectional view showing a cross-section of a device for measuring brain signals according to some embodiments of the inventive concept. For simplicity of description, descriptions overlapping those of the device for measuring brain signals described with reference to FIG. 1 will be omitted.

Referring to FIG. 25, a plurality of pads 105 may be disposed within an electrode substrate 100.

Each of the plurality of flexible electrodes 120 may include a flexible polymer 122 extending in a third direction D3 of the electrode substrate 100, the plurality of sensing portions 140 disposed on the flexible polymer 122, and lines 125 disposed on the flexible polymer 122 and electrically connected to the plurality of sensing portions 140.

The flexible polymer 122 may have a thickness 122W of, for example, about 5 μm to about 15 μm in a first direction D1. The flexible polymer 122 may include, for example, at least any one selected from the group consisting of polyurethane, silicone, polyisoprene, polyolefin, polyethylene, polypropylene, polyvinyl chloride, ethylene-vinyl acetate, polybutadiene, polyoxymethylene, polytetrafluoroethylene, polyimide, nylon, polycarbonate, polypyrrole, and polyaniline.

The plurality of sensing portions 140 may be spaced apart in a third direction D3. Accordingly, various regions of the brain may be measured. The lines 125 may extend in the third direction D3 and thus electrically connect the plurality of sensing portions 140 to each other. The lines 125 may electrically connect the plurality of sensing portions 140 and the plurality of pads 105 within the electrode substrate 100.

The other structure may be substantially the same as the device for measuring brain signals described with reference to FIG. 1.

Hereinafter, the inventive concept will be described through Examples.

### Example

Stearyl acrylate was prepared as a first compound. Urethane diacrylate was prepared as a second compound. The first compound and the second compound were mixed to form a first mixture. The first compound was in an amount of about 40 parts by weight with respect to 100 parts by weight of the first mixture. The second compound was in an amount of about 60 parts by weight with respect to 100 parts by weight of the first mixture. TMP-PA was prepared as a crosslinker. The crosslinker was in an amount of about 1part by weight with respect to 100 parts by weight of the first mixture. DMPA and benzophenone were prepared as photoinitiators. The DMPA was in an amount of about 1 part by weight with respect to 100 parts by weight of the first mixture. The benzophenone was in an amount of about 0.5 parts by weight with respect to 100 parts by weight of the first mixture. The crosslinker and the photoinitiators were mixed in the first mixture, and then the mixture was mixed in ethyl acetate to prepare a first solution.

Thereafter, ultraviolet light having a wavelength of about 365 nm was applied to the first solution to form a polymer.

### Evaluation Example: Mechanical Properties

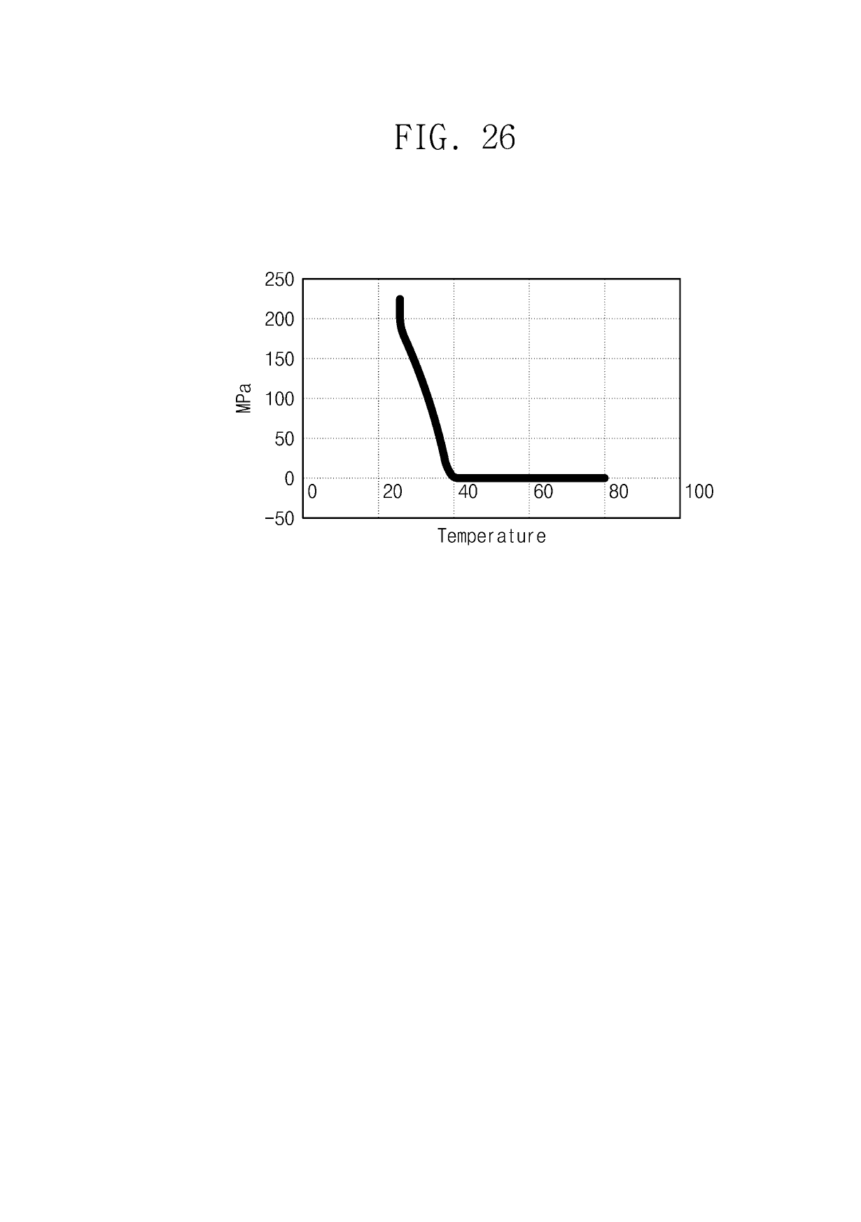

Storage modulus of the polymer according to Example was measured through a tensile test. The measurement results are shown in FIG. 26. Referring to FIG. 26, the polymer according to an embodiment was found to have a storage elastic modulus of about 100 MPa at a temperature of about 25 °C. In addition, the polymer was found to have a storage elastic modulus of about 1 MPa at a temperature of about 40 °C.

That is, it is determined that the polymer according to an embodiment of the inventive concept has a high storage elastic modulus at room temperature (about 25 °C) and a low storage elastic modulus at a temperature higher than body temperature (about 40 °C).

According to embodiments of the inventive concept, a device for measuring brain signals may have a flexible electrode and a polymer layer covering the flexible electrode disposed therein. The polymer layer may have high mechanical properties at a temperature lower than body temperature (e.g., room temperature), and may have reduced mechanical properties at or above body temperature. Accordingly, a flexible electrode having sufficient mechanical properties during insertion into the brain and having low mechanical properties after insertion and thus being flexibly deformable may be provided.

That is, after insertion into the brain, the electrode may be flexibly deformed according to the movement of the brain, thereby minimizing damage to brain tissue. In addition, contact between the brain tissue and the electrode may be minimized by the polymer layer and consequently damage to the brain tissue may be minimized, thereby obtaining brain signals and nerves.

In the above, embodiments of the inventive concept have been described with reference to the accompanying drawings, but those skilled in the art to which the inventive concept pertains may understand that various modifications and changes may be made to the inventive concept insofar as such modifications and changes do not depart from the spirit and scope of the inventive concept set forth in the claims to be described later. In addition, the embodiments disclosed in the inventive concept are not intended to limit the technical spirit of the inventive concept, and the scope of the disclosure is not to be limited by the above embodiments but by the claims and the equivalents thereof.

What is claimed is:

1. A device for measuring brain signals, the device comprising:
an electrode substrate comprising a first surface and a second surface opposing each other;
a plurality of flexible electrodes extending in a direction perpendicular to the first surface of the electrode substrate on the electrode substrate; and
a polymer layer covering at least a portion of a side surface of each of the plurality of flexible electrodes,
wherein each of the plurality of flexible electrodes comprises a plurality of sensing portions,
the polymer layer has greater mechanical properties at a temperature of 25 °C than at a temperature of 36 °C, and
the mechanical properties are storage modulus.

2. The device for measuring brain signals of claim 1, wherein the polymer layer has mechanical properties of 3 MPa or less at a temperature of 36 °C or greater.

3. The device for measuring brain signals of claim 1, wherein the polymer layer exposes the plurality of sensing portions.

4. The device for measuring brain signals of claim 1, wherein at a temperature of 25 °C to 36 °C, the polymer layer has greater mechanical properties than each of the plurality of flexible electrodes.

5. The device for measuring brain signals of claim 1, wherein the polymer layer has a thickness of 10 μm to 100 μm.

6. The device for measuring brain signals of claim 1, wherein the plurality of sensing portions disposed on each of the plurality of flexible electrodes are spaced apart in a direction perpendicular to the first surface of the substrate.

7. The device for measuring brain signals of claim 1, further comprising a plurality of electrode connection portions passing through the electrode substrate, and
each of the plurality of electrode connection portions is mechanically coupled to a corresponding flexible electrode among the plurality of flexible electrodes.

8. The device for measuring brain signals of claim 1, wherein a plurality of pads are disposed within the electrode substrate, and
the plurality of pads are electrically connected to the plurality of sensing portions.

9. The device for measuring brain signals of claim 1, wherein each of the plurality of flexible electrodes comprises at least any one selected from the group consisting of polypyrrole, polyaniline, graphene, carbon nanotubes, silver nanowires, and copper nanowires.

10. The device for measuring brain signals of claim 1, wherein each of the plurality of flexible electrodes comprises:
a flexible polymer extending in a direction perpendicular to the first surface of the electrode substrate;
a plurality of sensing portions disposed on the flexible polymer; and
a line disposed on the flexible polymer and electrically connected to the plurality of sensing portions.

11. The device for measuring brain signals of claim 1, wherein lengths of each of the plurality of flexible electrodes in the direction perpendicular to the first surface of the electrode substrate are different from one another.

12. A device for measuring brain signals, the device comprising:
an electrode substrate comprising a first surface and a second surface opposing each other;
a plurality of flexible electrodes extending in a direction perpendicular to the first surface of the electrode substrate on the electrode substrate; and
a polymer layer covering at least a portion of a side surface of each of the plurality of flexible electrodes,
wherein each of the plurality of flexible electrodes includes a plurality of sensing portions,
the polymer layer has a glass transition temperature of 34 °C to 40 °C, and
the polymer layer changes from crystalline to amorphous at or above the glass transition temperature.

13. The device for measuring brain signals of claim 12, wherein the polymer layer comprises:
a first repeating unit including an acrylate group (-COO-); and
a second repeating unit including a urethane group (-NH-CO-O-),
the first repeating unit amounts to 30 to 45 parts by weight with respect to 100 parts by weight of the polymer layer, and
the second repeating unit amounts to 55 to 70 parts by weight with respect to 100 parts by weight of the polymer layer.

14. The device for measuring brain signals of claim 12, wherein mechanical properties of the polymer layer at a temperature of 25 °C are 100 to 110 times greater than mechanical properties of the polymer layer at a temperature of 40 °C.

15. The device for measuring brain signals of claim 13, wherein:
the first repeating unit is derived from stearyl acrylate; and
the second repeating unit is derived from urethane diacrylate.

16. The device for measuring brain signals of claim 12, wherein the polymer layer has a thickness of 10 μm to 100 μm.

17. The device for measuring brain signals of claim 12, wherein each of the plurality of flexible electrodes has mechanical properties of 3 MPa or less.
